(19) United States
(12) Patent Application Publication
(10) Pub. No.: US 2023/0380929 A1
(43) Pub. Date: Nov. 30, 2023

(54) SURGICAL GUIDELINE MARKING LAYER, METHODS OF PRODUCING AND METHODS OF USING THEREOF

(71) Applicant: Chih Wei CHEN, Taipei City (TW)

(72) Inventor: Chih Wei CHEN, Taipei City (TW)

(21) Appl. No.: 18/297,023

(22) Filed: Apr. 7, 2023

Related U.S. Application Data
(60) Provisional application No. 63/328,369, filed on Apr. 7, 2022.

(51) Int. Cl. A61B 90/92; A61B 90/94

(52) U.S. Cl. CPC A61B 90/92; A61B 90/94; A61B 2090/373

(57) ABSTRACT

The present disclosure provides a bi-layer surgical guideline marking layer, including a first layer made of transparent material, a second layer made of transparent material, and the second layer is relatively arranged under the first layer. A first storage tank is arranged on the first same side of the first layer and the second layer. The present disclosure also provides a method for producing surgical guideline marking layer and a method for using it, so as to help surgeons shorten the operation time and improve the accuracy of the surgery position.

### CROSS-REFERENCE TO RELATED APPLICATION

This application claims priority to U.S. Provisional Application No. 63/328,369, filed Apr. 7, 2022, which is herein incorporated by reference in its entirety.

### BACKGROUND

### Field of Invention

The present disclosure relates to a surgical guidance marking layer used in surgery and its production and use methods to assist users in surgery.

### Description of Related Art

At present, in the traditional operation method, most of them mark the patient's anatomical features on the patient's body surface with a surgical marker pen at the moment of the operation as a reference for the position of the surgery. However, such a method cannot accurately present surgical information related to the surgery, especially the surgical planning path. For example, in general surgical operations, surgeons must use medical imaging equipment to confirm the relative relationship between the surgical instruments and the patient's physiological and anatomical features in space during the operation by scanning continuous X-ray images, that is, position and angle relation. This causes the surgeon to repeatedly look up to confirm the X-ray images, and then lower his head to confirm the operation site where the surgical instruments are located during the operation. Repeatedly switching attention increases the difficulty of the operation.

Therefore, in order to reduce the burden to the surgeons during the surgery and to improve the accuracy of the position of the surgery, its related technologies really need to be improved.

### SUMMARY

The present disclosure provides a bi-layer surgical guideline marking layer, the bi-layer surgical guideline marking layer comprises a first layer, a second layer, and a first receiving tank. The first layer is transparent material. The second layer is transparent material, and the second layer is relatively disposed under the first layer. The first receiving tank is disposed on a first same side of the first layer and the second layer.

In some embodiments, the bi-layer surgical guideline marking layer further comprises a second receiving tank. The second receiving tank is disposed on a second same side of the first layer and the second layer, and is disposed at an opposite side of the first receiving tank, an adjacent side of the first receiving tank, or the opposite side and two adjacent sides of the first receiving tank.

In some embodiments, the bi-layer surgical guideline marking layer further comprises a first permeable membrane and an access hole. The first permeable membrane is disposed between the first same side and the first receiving tank; and the access hole is disposed on the first layer.

In some embodiments, the bi-layer surgical guideline marking layer further comprises the first permeable membrane, a second permeable membrane, and the access hole. The first permeable membrane is disposed between the first same side and the first receiving tank. The second permeable membrane is disposed between the second receiving tank, the first layer and the second layer. The access hole is disposed on the first layer.

In some embodiments, the first layer displays a first color, the second layer displays a second color, the first color is different from the second color, when the first layer overlaps the second layer, the bi-layer surgical guideline marking layer displays a third color.

In some embodiments, the bi-layer surgical guideline marking layer further comprises at least one first instrument through hole penetrating the first layer and the second layer, and the first layer and the second layer is closely attached around the at least one first instrument through hole.

In some embodiments, the bi-layer surgical guideline marking layer further comprises at least one removable structure, at least one hardware adapter, at least one attaching device, at least one tactile structure, at least one grating structure, at least one body positioning mark, or at least one medical image layer is disposed on the first layer, under the first layer, on the second layer, under the second layer or a combination thereof.

In some embodiments, the bi-layer surgical guideline marking layer further comprises a foldable wire is disposed at opposite sides or all around of the first layer and the second layer.

In some embodiments, the bi-layer surgical guideline marking layer, further comprises at least one signal element is disposed on the foldable wire to sense an attached state of the bi-layer surgical guideline marking layer.

The present disclosure also provides a method of producing surgical guideline marking layer, comprising: providing at least one layer; a body positioning mark disposed at the at least one layer, as a reference for disposing the at least one layer at an object surface feature on an object surface; and a surgical guideline mark disposed on the at least one layer, as a guide reference for a surgical path, so as to obtain the surgical guideline marking layer.

In some embodiments, the method of producing surgical guideline marking layer further comprises performing a fixing way of the at least one layer, the fixing way comprises disposing an adhesive under the at least one layer, disposing a fixing device under the at least one layer, disposing a foldable wire on or under the at least one layer, or disposing at least one receiving tank around the at least one layer.

In some embodiments, the body positioning mark is an attaching device, the surgical guideline mark, a predetermining position, the object surface feature, a second continuous grid, a second discontinuous grid having at least one blank, at least one blank mark, at least one conducting blank mark relatively disposed at the at least one blank, or a combination thereof. In this disclosure, “object surface features” refers to a concave-convex structure of the internal structure of the object, the object surface also displays corresponding concave-convex feature, or the concave-convex structure of the object surface itself. For example, the convex and concave parts of the bones in the body let the body surface also displaying corresponding concave-convex features, the concave-convex features are also called anatomical features or physiological characteristics.

In some embodiments, the step of the body positioning mark disposed at the at least one layer, comprises: disposing a medical image imageable mark on the object surface, and reconstructing a curved surface of the object surface through a computer vision calculation, wherein the medical image imageable mark comprises a first continuous grid, a first discontinuous grid having at least one blank, or a combination thereof; and scanning a medical imaging for the medical image imageable mark together with an object of the object surface to produce the surgical guideline marking layer comprises the object surface feature, the second continuous grid, the second discontinuous grid having at least one blank, the at least one blank mark, or a combination thereof.

In some embodiments, the step of the body positioning mark disposed at the at least one layer, comprises: disposing a medical image imageable mark on the object surface, and reconstructing a curved surface of the object surface through a computer vision calculation, wherein the medical image imageable mark comprises a first discontinuous grid having at least one blank; and scanning a medical imaging for the medical image imageable mark together with an object of the object surface to produce the surgical guideline marking layer comprises the object surface feature, and the at least one blank mark, wherein the first discontinuous grid having at least one blank is a conductive substance; wherein the surgical guideline marking layer further comprises at least one conducting blank mark relatively disposed at the at least one blank, and disposed under the surgical guideline marking layer.

In some embodiments, the step of the body positioning mark disposed at the at least one layer comprises printing the body positioning mark on the at least one layer, wherein a material of the body positioning mark is printing ink or a material that to be imaged in medical image, wherein the body positioning mark comprises circle, polygon, or physiological characteristics.

In some embodiments, the method of producing surgical guideline marking layer, before the step of body positioning mark printed at the at least one layer, comprises: placing a medical image imageable mark on the object surface; and scanning the object surface having the medical image imageable mark, wherein the medical image imageable mark printed on at least one layer becomes the body positioning mark.

In some embodiments, the method of producing surgical guideline marking layer further comprises printing auxiliary information on the at least one layer, wherein the auxiliary information comprises a patient's medical image, a patient's identification data, a condition reminder, a surgery reminder information, or a combination thereof.

In some embodiments, the method of producing surgical guideline marking layer, wherein the surgical guideline mark comprises a visual characteristic, a tactile characteristic, a physical object characteristic, or a combination thereof; wherein the visual characteristic comprises a circle, a polygon, a pinhead, a line, a fillet, a surgical instrument copy shape, an implant copy shape, at least one grating structure with optical refraction, reflection, light concentrating effect, or a combination thereof; wherein the tactile characteristic comprises at least one directional protruded structure; wherein the physical object characteristic comprises a fixing device.

The present disclosure also provides a method of using surgical guideline marking layer, comprising: providing at least one surgical guideline marking layer obtained by the method of producing; fixing the surgical guideline marking layer on an object surface; and corresponding an identification mark or an object surface feature on the object surface to the body positioning mark and/or the surgical guide mark of the surgical guideline marking layer to position the surgical guideline marking layer.

In some embodiments, the step of fixing the surgical guideline marking layer on the object surface comprises using a fixing device, frictional force or static electricity of the surgical guideline marking layer, adhesive, or a foldable wire way to fix the surgical guideline marking layer on the object surface.

In some embodiments, the step of corresponding the identification mark or the object surface feature on the object surface to the body positioning mark and/or the surgical guide mark of the surgical guideline marking layer comprises: printing a predetermining position of the body positioning mark and the object surface feature on the surgical guideline marking layer, corresponding the object surface feature on the surgical guideline marking layer to the object surface feature on the object surface, and then disposing the body positioning mark at the predetermining position; scanning the object surface to obtain a medical image; and determining whether the distance between the body positioning mark in the medical image and the object surface feature in the medical image is the same as the distance between the body positioning mark placed on the surgical guideline marking layer and the object surface feature on the surgical guideline marking layer, if they are not the same, adjust the position of the surgical guideline marking layer until the same.

In some embodiments, the step of corresponding the identification mark or the object surface feature on the object surface to the body positioning mark and/or the surgical guide mark of the surgical guideline marking layer comprises: printing the object surface feature on the surgical guideline marking layer, and corresponding the object surface feature on the surgical guideline marking layer to the object surface feature on the object surface.

In some embodiments, the step of corresponding the identification mark or the object surface feature on the object surface to the body positioning mark and/or the surgical guide mark of the surgical guideline marking layer comprises: after scanning a medical image imageable mark image on the object surface to formed the body positioning mark on the surgical guideline marking layer; before removing the medical image imageable mark, marking the position of the medical image imageable mark on the object surface by using the identification mark.

In some embodiments, the method of using surgical guideline marking layer further comprises determining the attached degree of the surgical guideline marking layer on the object surface.

In some embodiments, the step of determining the attached degree of the surgical guideline marking layer on the object surface comprises using at least one grating structure disposed on the surgical guideline marking layer, wherein when the surgical guideline marking layer is completely attached to the object surface, the body positioning mark, the surgical guideline mark, or a combination thereof will be completely displayed by illuminating the grating structure with light.

In some embodiments, the step of determining the attached degree of the surgical guideline marking layer on the object surface, comprises: disposing a medical image imageable mark on the object surface, and reconstructing the curved surface of the object surface through a computer vision calculation, wherein the medical image imageable mark comprises a first continuous grid, a first discontinuous grid having at least one blank, or a combination thereof; scanning a medical imaging for the medical image imageable mark together with an object of the object surface to produce the surgical guideline marking layer comprises a patient's medical image, a second continuous grid, a second discontinuous grid having at least one blank, at least one blank mark, or a combination thereof; and disposing the surgical guideline marking layer on the object surface, wherein, when the second continuous grid is corresponding to the first continuous grid, the surgical guideline marking layer is determined to attach to the object surface; wherein, when the second discontinuous grid having at least one blank is corresponding to the first discontinuous grid having at least one blank, the surgical guideline marking layer is determined to attach to the object surface; or wherein, when the at least one blank mark is corresponding to the first discontinuous grid having at least one blank, the surgical guideline marking layer is determined attach to the object surface.

In some embodiments, the step of determining the attached degree of the surgical guideline marking layer on the object surface comprises: disposing a medical image imageable mark on the object surface, and reconstructing the curved surface of the object surface through a computer vision calculation, wherein the medical image imageable mark comprises a first continuous grid, a first discontinuous grid having at least one blank, or a combination thereof; scanning a medical imaging for the medical image imageable mark together with an object of the object surface to produce the surgical guideline marking layer comprises a patient's medical image, a second continuous grid, a second discontinuous grid having at least one blank, at least one blank mark, or a combination thereof; and disposing the surgical guideline marking layer on the object surface, wherein the first discontinuous grid having at least one blank is a conductive substance; wherein the surgical guideline marking layer further comprises at least one conductive blank mark correspondingly disposed at the at least one blank, and disposed under the surgical guideline marking layer; wherein, when the at least one conductive blank mark is electrically connected to the first discontinuous grid having the at least one blank, the surgical guideline marking layer is determined to attach to the object surface.

In some embodiments, the method of using surgical guideline marking layer, wherein the conductive blank mark comprises a first distance sensor communicating with a second distance sensor on a surgical instrument.

In some embodiments, the method of using surgical guideline marking layer further comprises determining whether a surgical information on the surgical guideline marking layer is consistent with the current state of the patient, if so, starting to use the surgical guideline marking layer.

In some embodiments, the method of using surgical guideline marking layer, wherein the step of determining whether a surgical information on the surgical guideline marking layer is consistent with the current state of the patient, if not, determining whether the object surface remains a medical image imageable mark produced in the previous surgical guideline marking layer, if not, regenerating another surgical guideline marking layer according to the surgical requirements.

In some embodiments, the method of using surgical guideline marking layer, the step of determining whether the object surface remains a medical image imageable mark produced in the previous surgical guideline marking layer, if so, determining whether a complete patient's medical image can be scanned at the moment, if not, regenerating the surgical guideline marking layer by scanning the current medical image of the patient, and using a computer vision calculations to update the medical image of the patient.

In some embodiments, the method of using surgical guideline marking layer, the step of determining whether a complete patient's medical image can be scanned at the moment, if so, determining whether the surgical information on the surgical guideline marker layer is necessary to redesign, if so, scanning the complete patient's medical image again, and regenerating the surgical guideline marking layer according to an operation requirements; if not, then using the currently scannable patient's medical image, and updating by a computer vision calculations to regenerate the surgical guideline marking layer.

### BRIEF DESCRIPTION OF THE DRAWINGS

Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion. The disclosure can be more fully understood by reading the following detailed description of the embodiment, with reference made to the accompanying drawings as follows.

FIG. 1 shows a schematic view of a bi-layer surgical guideline marking layer structure in some embodiments of the present disclosure.

FIG. 2 shows a schematic view of the bi-layer surgical guideline marking layer collocated with different instrument through holes in some embodiments of the present disclosure.

FIG. 3 shows a schematic view of disposing a removable structure on the bi-layer surgical guideline marking layer in accordance with some embodiments of the present disclosure.

FIG. 4 shows a schematic view of setting attaching devices and hardware adapters on the bi-layer surgical guideline marking layer in some embodiments of the present disclosure.

FIG. 5 shows a side view of a tactile structure disposed at the bi-layer surgical guideline marking layer in with some embodiments of the present disclosure.

FIG. 6 shows a side view of a visual feature grating structure disposed at the bi-layer surgical guideline marking layer in some embodiments of the present disclosure.

FIG. 7 shows a schematic view of disposing body positioning marks that to be imaged in medical imaging in the bi-layer surgical guideline marking layer in some embodiments of the present disclosure.

FIG. 8 shows some embodiments of this disclosure in the bi-layer surgical guideline marking layer, a schematic view of generating body positioning mark positions based on the obvious physiological characteristics of the patient's medical images.

FIG. 9 shows a schematic view of the body positioning mark predetermined position of some embodiments of the present disclosure in the pre-planned placement of the bi-layer surgical guideline marking layer.

FIG. 10 shows a schematic view of medical images of patients printed on the bi-layer surgical guideline marking layer in some embodiments of the present disclosure.

FIG. 11 shows a schematic view about a foldable wire disposed around the bi-layer surgical guideline marking layer or a signal element disposed on the foldable wire in some embodiments of the present disclosure.

FIG. 12 shows a perspective view of a fixing device in some embodiments of the present disclosure.

FIG. 13 shows a three-dimensional schematic view of different examples of the fixing device in some embodiments of the present disclosure.

FIG. 14 shows cross-sectional views of different examples of the fixing devices in some embodiments of the present disclosure.

FIG. 15 shows a three-dimensional schematic view of different examples of the fixing devices in some embodiments of the present disclosure.

FIG. 16 shows cross-sectional views of different examples of the fixing devices in some embodiments of the present disclosure.

FIG. 17 shows three-dimensional section view of different embodiments of the fixing devices in some embodiments of the present disclosure.

FIG. 18 shows a schematic illustration of an example without an instrument guide layer in the fixing device in some embodiments of the present disclosure.

FIG. 19 shows a schematic top view of an example of the second instrument through hole on the parallel extended structure of the fixing device in some embodiments of the present disclosure.

FIG. 20 shows the schematic about the fixing device and a surgical assistive device are integrally formed in some embodiments of the present disclosure.

FIG. 21A to FIG. 21B show a schematic view of the examples about combination and decomposition of the fixing device and the surgical assistive device in some embodiments of the present disclosure.

FIG. 22 shows a schematic view of the fixing device, a flexible adapter and the surgical assistive device are used together in some embodiments of the present disclosure.

FIG. 23 shows a schematic view about the parallel extended structure and a mono-layer surgical guideline marking layer are integrally formed in the fixing device in some embodiments of the present disclosure.

FIG. 24A to FIG. 24B show a schematic view about the combination and decomposition of the fixing device and the mono-layer surgical guideline marking layer in some embodiments of the present disclosure.

FIG. 25 is a flow chart illustrating a method of producing of surgical guideline marking layer.

FIG. 26A to FIG. 26B shows a schematic view of surgical guideline marking layer with body positioning mark.

FIG. 27 shows a schematic view of using body positioning markers that to be imaged in medical imaging.

FIG. 28 shows a schematic view of producing medical image imageable mark in surgical guideline marking layer.

FIG. 29A to FIG. 29B show a schematic view of surgical guideline marking layers in the form of visual features in accordance with some embodiments of the present disclosure.

FIG. 29C shows a schematic view of surgical guideline marking layers in the form of tactile features in some embodiments of the present disclosure.

FIG. 29D shows a schematic view about physical object features of the fixing device, the surgical guideline marking layer, the flexible adapter, and the surgical assistive device used together in some embodiments of the present disclosure.

FIG. 29E shows a schematic view of using the attaching devices and the hardware adapters at surgical guideline marking layer in some embodiments of the present disclosure.

FIG. 30 is a flow chart illustrating a method of using surgical guideline marking layer in some embodiments of the present disclosure.

FIG. 31 to FIG. 33 are schematic views of using different fixing ways to fix the surgical guideline marking layer on the patient in some embodiments of the present disclosure.

FIG. 34 shows a schematic view of pre-planning and placing body positioning mark predetermined position on the surgical guideline marking layer in some embodiments of the present disclosure.

FIG. 35 shows a schematic view of a grating structure disposed at surgical guideline marking layer in accordance with some embodiments of the present disclosure.

FIG. 36 shows a schematic view of using at least one grating structure to determine the attached degree of the surgical guideline marking layer in some embodiments of the present disclosure.

FIG. 37A shows a schematic view of the surgical guideline marking layer that has been completely attached to the surface of the patient in some embodiments of the present disclosure.

FIG. 37B shows a schematic view of the complete image information in FIG. 37A.

FIG. 38A shows a schematic view of the surgical guideline marking layer that has not been completely attached to the surface of the patient in some embodiments of the present disclosure.

FIG. 38B shows a schematic view of the not complete image information in FIG. 38A.

FIG. 39A shows a schematic view of disposing the first continuous grid of identifiable mark on the object surface in some embodiments of the present disclosure.

FIG. 39B shows a schematic view of the surgical guideline marking layer is producing after scanning medical images in FIG. 39A.

FIG. 40A shows a schematic view of disposing the first discontinuous grid of identifiable mark on the object surface in some embodiments of the present disclosure.

FIG. 40B shows a schematic view of the surgical guideline marking layer is producing after scanning medical images in FIG. 40A.

FIG. 41 shows a schematic view of the medical image scanned in FIG. 40A.

FIG. 42 shows a schematic view of the corresponding relationship between the patient medical image layer and the blank mark of surgical guideline marking layer in some embodiments of the present disclosure.

FIG. 43 shows a schematic view of the connection of the surgical guideline marking layer disposed on the body surface in some embodiments of the present disclosure.

FIG. 44 shows a schematic view of a triangulation measurement that can be performed with the first distance sensor and the second distance sensor on the surgical instrument in conjunction with the surgical guideline marking layer in accordance with some embodiments of the present disclosure.

FIG. 45A shows a schematic view of using the auxiliary block together with the surgical guideline marking layer to assist the surgical instrument in some embodiments of the present disclosure.

FIG. 45B shows a schematic view of using the auxiliary block together with the surgical guideline marking layer to assist the surgical instrument in another embodiments of the present disclosure.

FIG. 46 is a flow chart of a method of using surgical guideline marking layer following in FIG. 30 according to some embodiments of the present disclosure.

### DETAILED DESCRIPTION

The following disclosure provides detailed description of many different embodiments, or examples, for implementing different features of the provided subject matter. These are, of course, merely examples and are not intended to limit the disclosure but to illustrate it. In addition, various embodiments disclosed below may combine or substitute one embodiment with another, and may have additional embodiments in addition to those described below in a beneficial way without further description or explanation. In the following description, many specific details are set forth to provide a more thorough understanding of the present disclosure. It will be apparent, however, to those skilled in the art, that the present disclosure may be practiced without these specific details.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising”, or “includes” and/or “including” or “has” and/or “having” when used in this specification, specify the presence of stated features, regions, integers, steps, operations, elements, and/or components, but do not preclude the presence or addition of one or more other features, regions, integers, steps, operations, elements, components, and/or groups thereof.

The mode of bi-layer surgical guideline marking layer.

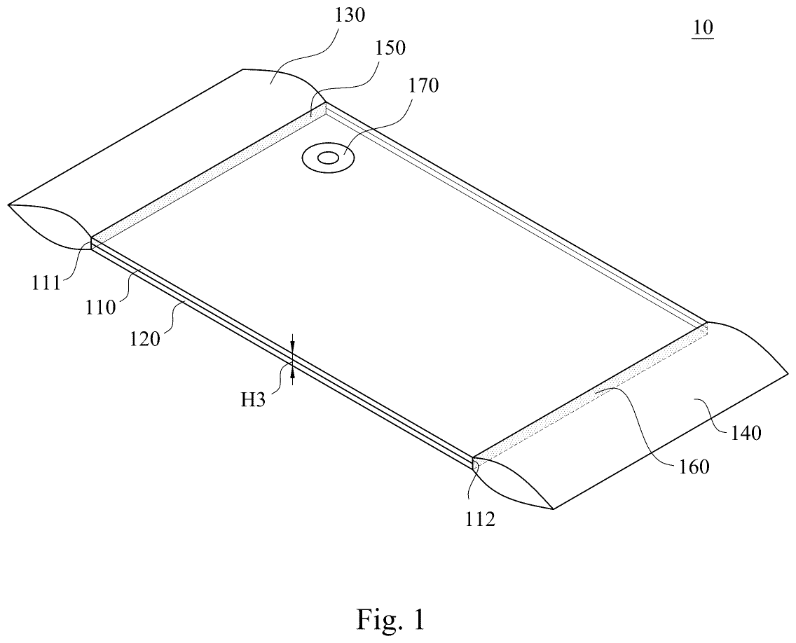

Please refer to FIG. 1, FIG. 1 shows a schematic of a bi-layer surgical guideline marking layer 10 structure in some embodiments of the present disclosure. The bi-layer surgical guideline marking layer 10 including a first layer 110, a second layer 120, a first receiving tank 130, a second receiving tank 140, a first permeable membrane 150, a second permeable membrane 160, and an access hole 170.

The first layer 110 is a transparent material. In some examples, the first layer 110 can be transparent and colorless or colored, such as blue or yellow.

The second layer 120 relatively disposed under the first layer 110. In some examples, the second layer 120 is a transparent material. Specifically, when the first layer 110 is blue, the second layer 120 can be yellow, and the color is green when overlapped; or when the first layer 110 is yellow, the second layer 120 can be blue, and the color is also green when overlapped. For another example, when the first layer 110 is transparent and the second layer 120 is red, the color is different from the original red when overlapped, such as light red; when the first layer 110 is transparent, the second layer 120 is blue, and then the color is light blue when overlapped. In other words, the combination of the third color can be applied through the combination of the first color and the second color, and is not limited to the colors listed above, so as to achieve the determination of bi-layer surgical guideline marking layer 10 that has been attached to the patient's body surface. In some embodiments, the overlay recognition of the first layer 110 and the second layer 120 includes, but is not limited to color, typeface, line or pattern to identify whether the first layer 110 and the second layer 120 overlapped and attached to the patient's body surface.

The first receiving tank 130 is disposed on a first same side 111 of the first layer 110 and the second layer 120.

The second receiving tank 140 is disposed on a second same side 112 of the first layer 110 and the second layer 120, and disposed at an opposite side of the first receiving tank 130. The weight generated by the liquid contained in the two sides of the receiving tanks 130 can make the bi-layer surgical guideline marking layer 10 generate tension, making the bi-layer surgical guideline marking layer 10 more attached to the patient's body surface.

In some examples, bi-layer surgical guideline marking layer 10 includes, but is not limited to the first receiving tank 130 is disposed at the first same side 111, the second receiving tank 140 is disposed at the second same side 112, having the first receiving tank 130 and the second receiving tank 140 at the same time are respectively disposed at the first same side 111 and the second same side 112, having the first receiving tank 130 is disposed at the first same side 111 and the second receiving tank 140 is disposed at the adjacent side of the first receiving tank 130 at the same time, or having the first receiving tank 130 disposed at the first same side 111 and the second receiving tank 140 disposed at the adjacent two sides of the first receiving tank 130 and the second same side 112 at the same time (that is, the first receiving tank 130 and the second receiving tank 140 are disposed around the bi-layer surgical guideline marking layer 10).

The first permeable membrane 150 is disposed between the first same side 111 and the first receiving tank 130. With the disposing of the first permeable membrane 150, the fluid that has penetrated into the first receiving tank 130, such as a liquid, will not seep back under an atmospheric pressure, and can only seep back into the space between the first layer 110 and the second layer 120 by applying pressure. The liquid includes, but is not limited to, a hydrophilic liquid (such as water), a hydrophobic liquid (such as oil), or a combination thereof. In some examples, the fluid is a gas.

The second permeable membrane 160 is disposed between the second same side 112 and the second receiving tank 140. In some examples, the second permeable membrane 160 is disposed between the second receiving tank 140, and the first layer 110 and the second layer 120 according to different positions of the second receiving tank 140. With the disposing of the second permeable membrane 160, the fluid that has penetrated into the second receiving tank 140, such as a liquid, will not seep back under an atmospheric pressure, and can only seep back into the space between the first layer 110 and the second layer 120 by applying pressure. The liquid includes, but is not limited to, a hydrophilic liquid (such as water), a hydrophobic liquid (such as oil), or a combination thereof. In some examples, the fluid is a gas.

The access hole 170 is disposed on the first layer 110, in which when the access hole 170 injects fluid, an receiving space is formed between the first layer 110 and the second layer 120 to accommodate the fluid, and then when the fluid in the receiving space is scraped off to both sides by a scraper, tension can be generated in the bi-layer surgical guideline marking layer 10, making the bi-layer surgical guideline marking layer 10 more attached to the patient's body surface. The fluid will permeate through the first permeable membrane 150 to the first receiving tank 130, and the fluid will also permeate through the second permeable membrane 160 to the second receiving tank 140. In some examples, when the fluid in the receiving space of the bi-layer surgical guideline marking layer 10 is scraped off, the first layer 110 and the second layer 120 will be overlapped, and the overlapped third color will appear, make sure that the bi-layer surgical guideline marking layer 10 has been attached to the patient's body surface. In some examples, when the bi-layer surgical guideline marking layer 10 is not needed temporarily, the access hole 170 can be used to drain the fluid for storage. When repeatedly placing the bi-layer surgical guideline marking layer 10, the fluid can be refilled through the access hole 170, and the fluid can also be drained through the access hole 170, shortening the time for placing the bi-layer surgical guideline marking layer 10.

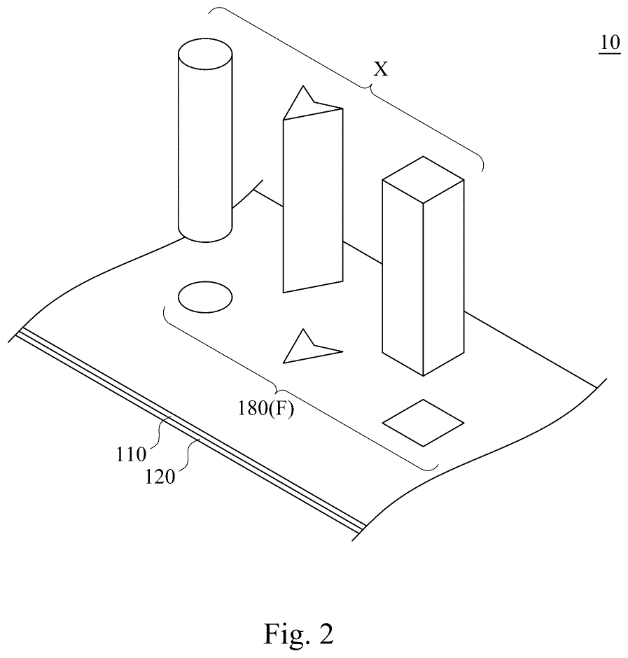

External processing methods can be used to make the surface of the bi-layer surgical guideline marking layer 10 having the surgical information of the surgery. The external processing methods include: printing, assembly or other processing methods. The surgical information refers to any content that can provide reference information for surgeons or other nursing staff during surgery. The surgical information includes, but is not limited to patient medical imaging, for example: computer tomography (CT), magnetic resonance imaging (MM), X-ray, ultrasound, thermal imaging etc., body positioning mark 205, surgical guideline mark F (as shown in FIG. 2), patient identification data, disease reminder, and surgery reminder information, or one or any combination thereof.

In some examples, if the surgical information processing method on the surface of the bi-layer surgical guideline marking layer 10 is printing, then the surface of the bi-layer surgical guideline marking layer 10 printed on the patient's body surface is less likely to be abrasion due to external force friction, resulting in the surgical information on the surface of bi-layer surgical guideline marking layer 10 disappearing. For example, if the manual information is printed on the surface of the first layer 110, when the surgical instrument X (as shown in FIG. 2) pierces through the surface of the first layer 110, the accompanying friction will cause abrasion of the surgery information. Therefore, printing the surgery information on the lower surface of the first layer 110, the upper surface of the second layer 120, or the lower surface of the second layer 120 (that is, the side in contact with the patient's surface) can avoid the abrasion of the printing of surgery information.

Please refer to FIG. 1 again, the thickness H3 of the bi-layer surgical guideline marking layer 10 must not affect the puncture resistance, so that the surgical instrument X (as shown in FIG. 2) can be punctured the first layer 110 of the bi-layer surgical guideline marking layer 10 to the second layer 120 of the bi-layer surgical guideline marking layer 10. In some examples, the thickness H3 of the bi-layer surgical guideline marking layer 10 can be 0.01 millimeter (mm) to 1 mm, so as not to be too thin and difficult to operate. For example: the bi-layer surgical guideline marking layer 10 is unable to fit the patient's body surface due to wrinkles, thereby affecting the accuracy; or the bi-layer surgical guideline marking layer 10 is too thick to be not easily bent.

The material of the bi-layer surgical guideline marking layer 10 needs to be soft and bendable, but not malleable, so that the bi-layer surgical guideline marking layer 10 can be attached to the patient's body but is not easy to be deformed due to pulling or other tension. In some examples, the material can be polyurethane (PU), thermoplastic polyurethane (TPU), silicone or other flexible materials. In addition, the second layer 120 of the bi-layer surgical guideline marking layer 10 can be processed to attach adhesive, or use a viscous material itself. And when the second layer 120 of the bi-layer surgical guideline marking layer 10 does not have any stickiness, the second layer 120 can be attached to the patient's body surface by the material such as TPU's own friction or electrostatic attraction.

Please refer to FIG. 2, FIG. 2 shows the bi-layer surgical guideline marking layer 10 collocated with different instrument through holes in some embodiments of the present disclosure. In FIG. 2 to FIG. 11 and FIG. 23 to FIG. 24B, structures such as the first receiving tank 130, the second receiving tank 140, the first permeable membrane 150, the second permeable membrane 160, and the access hole 170 are omitted to make the drawing simple. The bi-layer surgical guideline marking layer 10 further includes at least one first instrument through hole 180. The first instrument through hole 180 runs through the first layer 110 and the second layer 120, and the first layer 110 and the second layer 120 around at least one first instrument through hole 180 are in close contact with each other. In some examples, the size and position of the first instrument through hole 180 can be relatively matched with the first instrument through hole 180 based on the surgical instrument X feature, for example: the device diameter, shape, and the entry point of the surgical site are designed, and the characteristics of the device are designed as the path guidance of the surgical instrument X on the patient's body surface entry point. In the worth noting that when the bi-layer surgical guideline marking layer 10 has the characteristics of first instrument through hole 180, there is no bi-layer structure at the first instrument through hole 180; that is, the first layer 110 and the second layer 120 are closely attached to the periphery of the first instrument through hole 180 (e.g. thermal compression bonding). In some examples, the first instrument through hole 180 can be designed according to the surgical instrument X to be non-directional or directional. In other words, when the first instrument through hole 180 has further directional features, the shape of the first instrument through hole 180 can be an irregular polygonal outline, that is, the directional first instrument through hole 180 can also guide the path and angle of the surgical instrument X on the patient's body surface. In some examples, when the bi-layer surgical guideline marking layer does not have the feature of first instrument through hole 180, partial bi-layer surgical guideline marking layer 10 can be bonded by thermal compression to form at least one single-layer block. Bonding at least one layer of region blocks is now defined as a guide, the described pattern includes, but is not limited to the technical instrument copy shape F1, implant copy shape F2 or other inter-matching features.

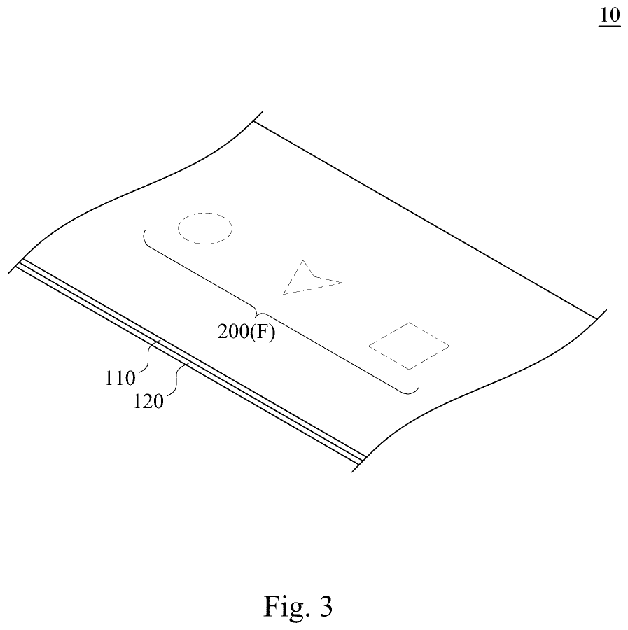

Please refer to FIG. 3, FIG. 3 shows a schematic view of disposing a removable structure 200 on the bi-layer surgical guideline marking layer 10 in accordance with some embodiments of the present disclosure. In some examples, the bi-layer surgical guideline marking layer 10 further includes at least one removable structure 200. Bi-layer surgical guideline marking layer 10 can have one or more removable structure 200, the one or more removable structure 200 can be formed by pre-cutting the dotted line on the bi-layer surgical guideline marking layer 10. The size and position of the removable structure 200 can be based on the characteristics of the surgical instrument X, for example, the instrument diameter, shape, and entry point of the surgical site are designed as a path guide for the entry point of the surgical instrument X on the patient's body surface.

It should be understood that only the first instrument through hole 180 and the removable structure 200 on the bi-layer surgical guideline marking layer 10 in FIG. 2 and FIG. 3 are different in different forms, all the features mentioned in the descriptive paragraph of FIG. 2 can also be display in the example of FIG. 3. For example, the first layer 110 around the removable structure 200 closely contacts with the second layer 120.

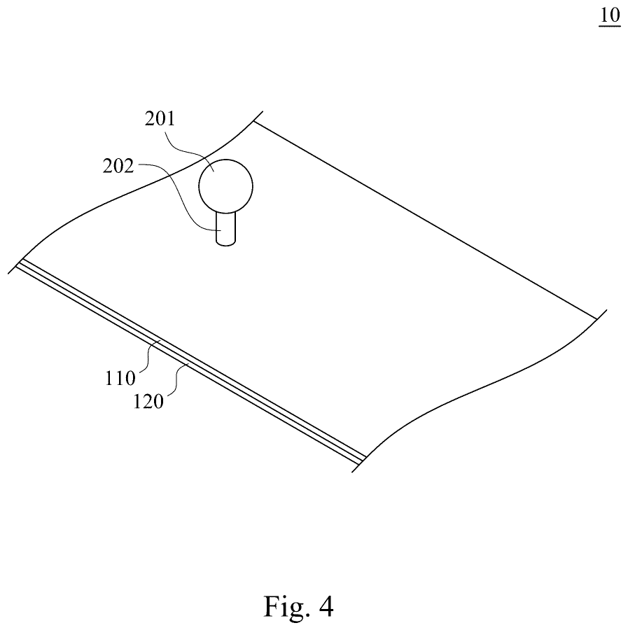

Please refer to FIG. 4, FIG. 4 shows a schematic view of disposing attaching devices 201 and hardware adapters 202 on the bi-layer surgical guideline marking layer 10 in some embodiments of the present disclosure. In some examples, bi-layer surgical guideline marking layer 10 further includes at least one attaching device 201 and hardware adapter 202 disposed on the first layer 110. In some examples, bi-layer surgical guideline marking layer 10 can include a hardware adapter 202, and the hardware adapter 202 can be used to connect any attaching device 201 wanting to be attached to the bi-layer surgical guideline marking layer 10. The attaching device 201 is for example, body positioning mark 205, trackable device, assembly accessories, etc. The trackable device includes, but is not limited to an optical tracking marker ball, a radio frequency (RF) device, etc.

The hardware adapter 202 can be connected to the bi-layer surgical guideline marking layer 10 by using any processing methods such as snap-in buckle, sticking, thread, interlayer, etc., and the hardware adapter 202 can have the characteristics of the attaching device 201, making the way easier for users to assemble. For example, the connection between the hardware adapter 202 and the attaching device 201 can be a copy shape structure.

The bi-layer surgical guideline marking layer 10 can contain different types of surgical information. The following paragraphs illustrate an example when the surgical information is the surgical guideline marking F. Generally speaking, a surgeon will scan at least one medical image for a patient before the operation, and the medical image includes, but is not limited to CT, MRI, X-ray, ultrasound, thermal image, etc. After scanning the medical image, the surgeon will plan the suitable surgical method and location based on the medical image, in which the planning method includes, but is not limited to planning with the existing surgical planning system/software, and the surgical guideline mark F function is to mark the surgical information planned by the surgeon before the surgery bi-layer surgical guideline marking layer 10, and serve as a guiding reference for the surgical path during the surgery.

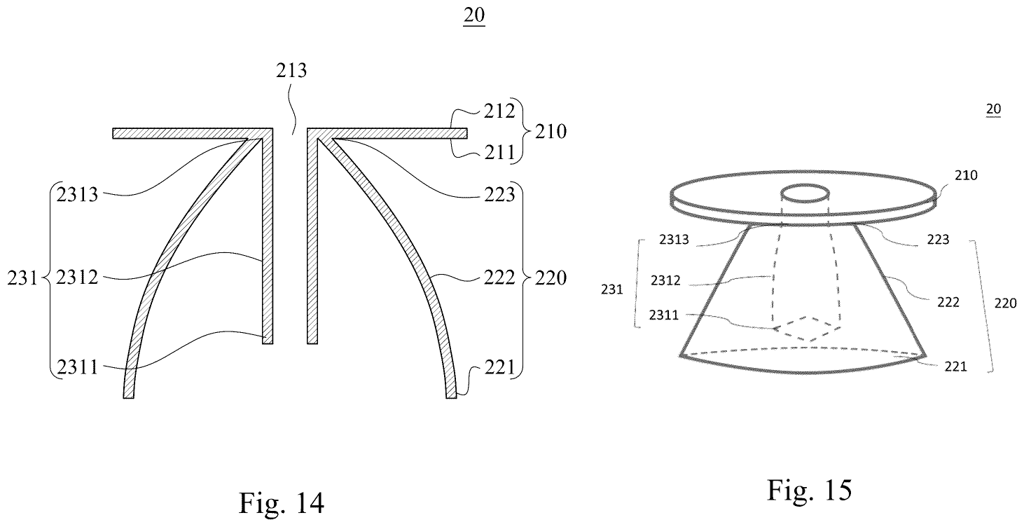

Surgical guideline mark F can be different types of features including, but not limited to: visual features, such as: image features, medical imaging features, grating features, tactile features, physical object features, etc., user can choose the above-mentioned different forms one or any combination of them can be used. Different types of features can apply different types of processing methods to combine the surgical guideline mark F with the bi-layer surgical guideline marking layer 10. Processing methods include, but are not limited to: printing, snap-in buckle, magnetic attraction, integral forming processing, etc. The characteristics of the described physical object features indicate that other physical object structures are used to connect with the bi-layer surgical guideline marking layer 10 as the surgical guide marker F. For example: the second instrument through hole 213 on the fixing device 20 mentioned above or its peripheral accessories is used as the surgical guideline mark F (as shown in FIG. 12 to FIG. 24B).

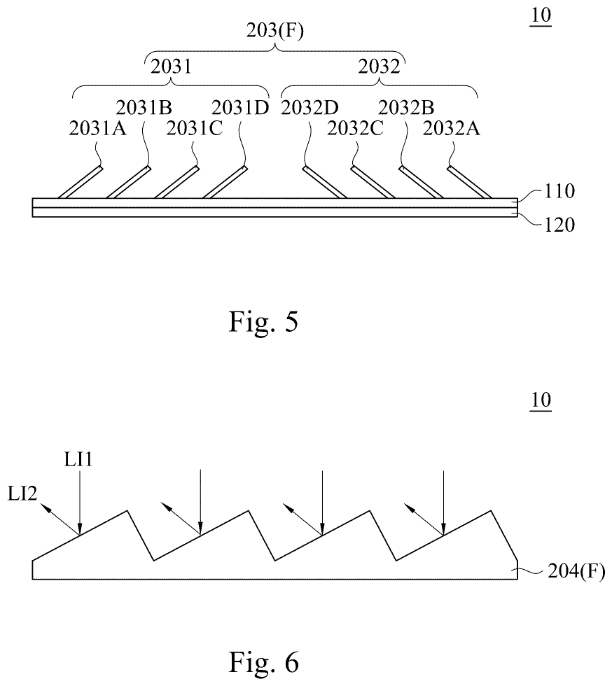

Please refer to FIG. 5, FIG. 5 shows a side view of a tactile structure 203 disposed at the bi-layer surgical guideline marking layer 10 in with some embodiments of the present disclosure. In some examples, bi-layer surgical guideline marking layer further includes at least one tactile structure 203 disposed on the first layer 110. The example illustrates the use of tactile features as surgical guideline mark F. In this example, the user can determine the position of the surgical guideline mark F through the tactile. For example, FIG. 5 includes a first direction tactile structure 2031 facing the first direction (such as first direction tactile structure 2031A, first direction tactile structure 2031B, first direction tactile structure 2031C, first direction tactile structure 2031D), and a second direction tactile structure 2032 facing the second direction (such as second direction tactile structure 2032A, second direction tactile structure 2032B, second direction tactile structure 2032C, second direction tactile structure 2032D) are protruded structures in different directions, users can feel the junction of protruded structures with different directions by touching, that is the junction of the first direction tactile structure 2031D and the second direction tactile structure 2032D, the junction is the reference position of the surgery path.

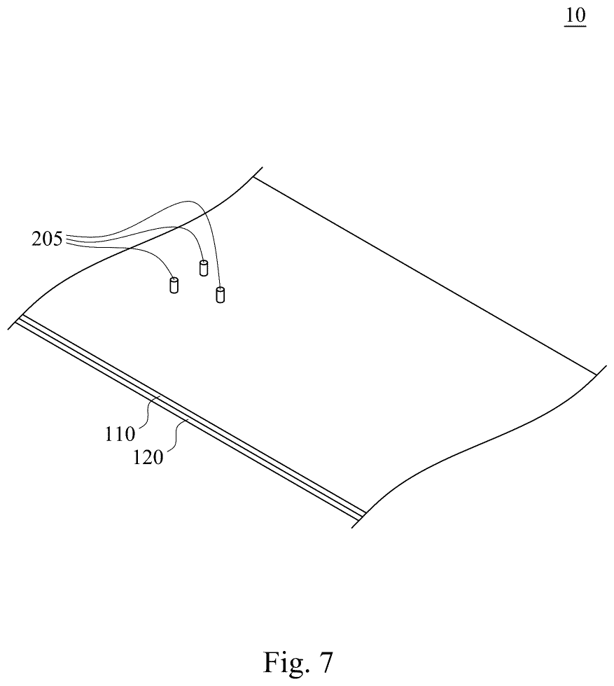

Please refer to FIG. 6, FIG. 6 shows a side view of a visual feature grating structure 204 disposed at the bi-layer surgical guideline marking layer 10 in some embodiments of the present disclosure. In some examples, the bi-layer surgical guideline marking layer 10 further includes at least one grating structure 204. The example illustrates the use of raster cues of visual features as surgical guideline mark F. In this example, light incidence path LI1 and the light reflection path LI2 are designed according to the angle of the instrument in the surgical planning, and when the grating structure 204 is used in the surgery, the user's line of sight must be the same as the light reflection path LI2 to see the complete surgical guideline mark F, and the user must keep the angle of the instrument consistent with the light reflection path LI2 as much as possible. Optical refraction/reflection/light spotting effects can also be used in conjunction with surgical instrument designs, for example, the direction of the grating is designed as the direction of the surgical planning path, and a visible light source (such as laser light) is designed on the surgical instrument, when the direction and position of the surgical instrument overlap with the direction and position of the surgical planning path, surgical instrument can reflect a complete and clear surgical planning path to serve as a reminder for surgical instrument.

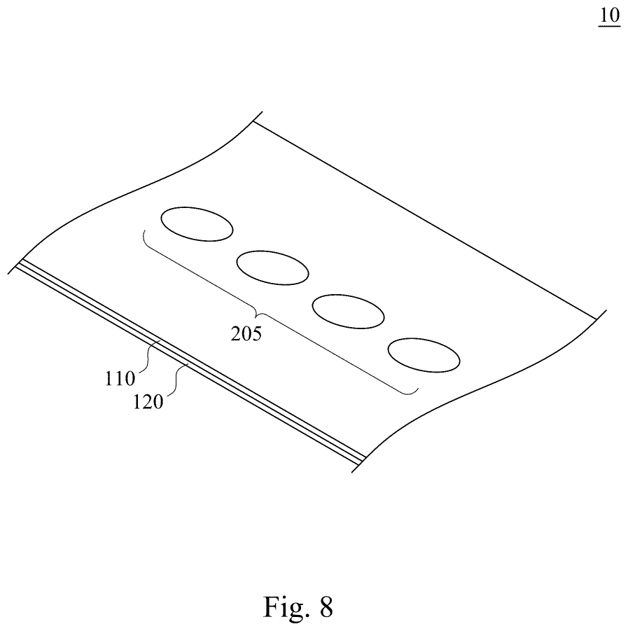

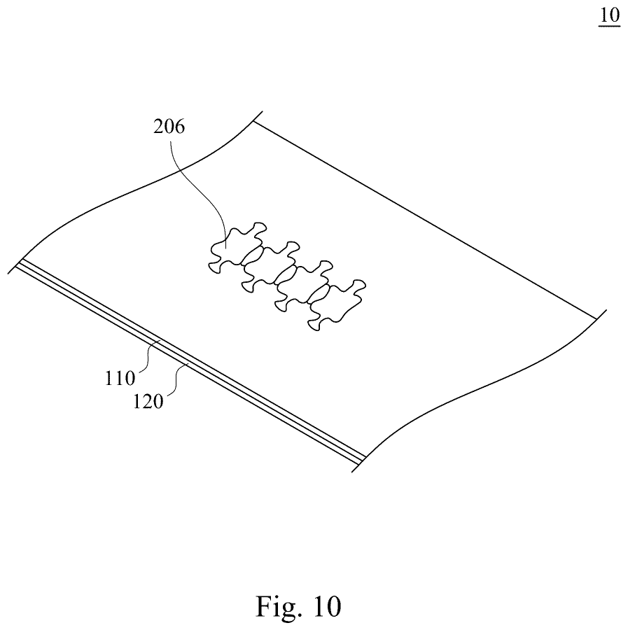

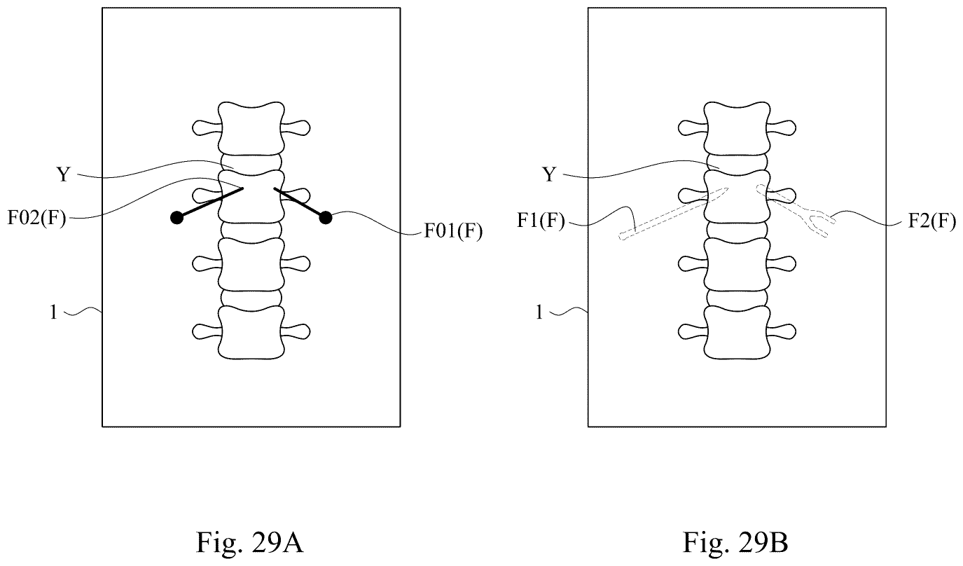

Body positioning mark 205 can be displayed in different forms, for sample: printed on the bi-layer surgical guideline marking layer 10 (as shown in FIG. 7), disposed the body positioning mark 205 that to be imaged in medical imaging on bi-layer surgical guideline marking layer 10 (as shown in FIGS. 4 and 9), scanned the medical image imageable mark 2053 with the patient before the operation (as shown in FIG. 27), etc. Different forms of displayed methods can be used in any combination, and no matter which displayed method is used, the use should be noted that the position of the body positioning mark 205 must avoid the actual part of the patient that needs surgery. The bi-layer surgical guideline marking layer 10 can include different types of surgical information. The following paragraphs illustrate the example when the surgical information is body positioning mark 205. The body positioning mark 205 can be used as a reference during the surgery to assist the surgeon in placing the bi-layer surgical guideline marking layer 10 on the patient's body surface at a preset position.

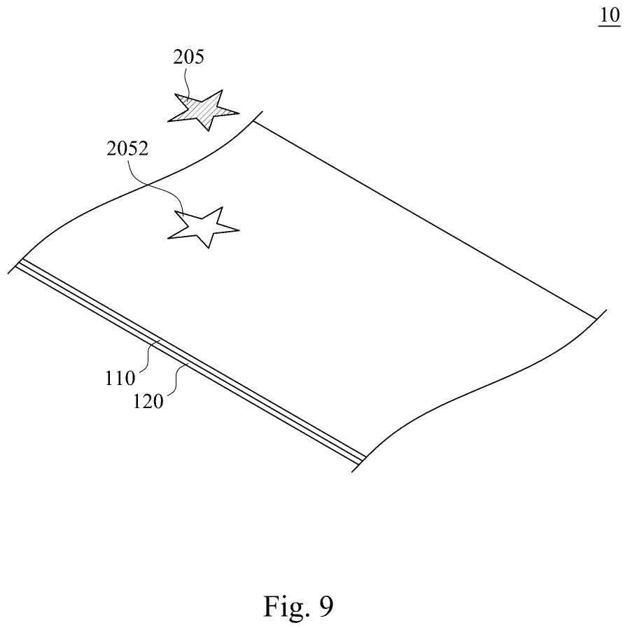

FIG. 7 and FIG. 8 are examples that the body positioning mark 205 is printed on the bi-layer surgical guideline marking layer 10. In these examples, the printed materials can be normal printed materials or printed materials that to be imaged in medical images including, but are not limited to: CT, MM, X-ray, ultrasound, thermal images, etc. When using printed materials that to be imaged in medical imaging, the characteristics of the printed materials can be adjusted according to the patient's physiological characteristics Y. For example, when using printed materials that to be imaged by X ray, the concentration of the X ray body positioning mark 205 printed material can be determined according to the bone density of the patient so that the bone image of the patient can be distinguished from the image of the X-ray image imageable marker. For example: patients with higher bone density use higher concentrations of X-ray imageable materials. In this way, when scanning X-ray images during the surgery, the X-ray image is possible to clearly distinguish the patient's bone images from the X-ray imageable marker images.

Please refer to FIG. 7 first, FIG. 7 shows a schematic view of disposing body positioning marks 205 that to be imaged in medical imaging in the bi-layer surgical guideline marking layer 10 in some embodiments of the present disclosure. In some examples, the bi-layer surgical guideline marking layer 10 further includes at least one body positioning mark 205 that to be imaged in medical imaging, disposed on the bi-layer surgical guideline marking layer 10, and printed on any one of the bi-layer surgical guideline marking layer 10 that does not block the location of the surgical site. The body positioning mark 205 can be in any pattern, for example: square, circle, arbitrary polygon, etc.

The body positioning mark 205 can be produced by the obvious physical characteristics Y of the patient's medical image. The physical characteristics Y include, but are not limited to: IC (iliac crest), posterior superior iliac spine (PSIS), spinous process any one or combination of thereof.

Please refer to FIG. 8, FIG. 8 shows some embodiments of this disclosure in the bi-layer surgical guideline marking layer 10, a schematic view of generating body positioning mark 205 positions based on the obvious physiological characteristics of the patient's medical images. In some examples, the bi-layer surgical guideline marking layer 10 further includes at least one pre-planned body positioning marker 205 disposed at the bi-layer surgical guideline marking layer 10. The body positioning mark 205 is generated according to the spinous process of the patient's medical image, and printed on the bi-layer surgical guideline marking layer 10. In this example, the surgeon can use palpation to quickly locate the body positioning mark 205 on the patient's body surface (such as palpation and touch to find the surface features of the object, such as physiological characteristics Y). Furthermore, if the body positioning mark 205 is printed by a medical imaging material, the surgeon can adjust the position of the bi-layer surgical guideline marking layer 10 on the patient's body surface by scanning intraoperative medical images after the initial placement.

In another example, the body positioning mark 205 can also be one or more devices that to be imaged in medical images, the medical images includes, but is not limited to: CT, MM, X-ray, ultrasound, thermal image etc.

Please refer to FIG. 9, FIG. 9 shows a schematic view of the body positioning mark 205 predetermined position 2052 of some embodiments of the present disclosure in the pre-planned placement of the bi-layer surgical guideline marking layer 10. The body positioning mark 205 can be placed directly on bi-layer surgical guideline marking layer 10. In some examples, the characteristics of the body positioning mark 205 can further be printed in advance at the predetermined position 2052 where the body positioning mark 205 is to be placed on the bi-layer surgical guideline marking layer 10. The characteristics of the body positioning mark 205 can be the copy shape or others related characteristics of the body positioning mark 205, so that the user can quickly place the body positioning mark 205 in position according to its features when placing the body positioning mark 205.

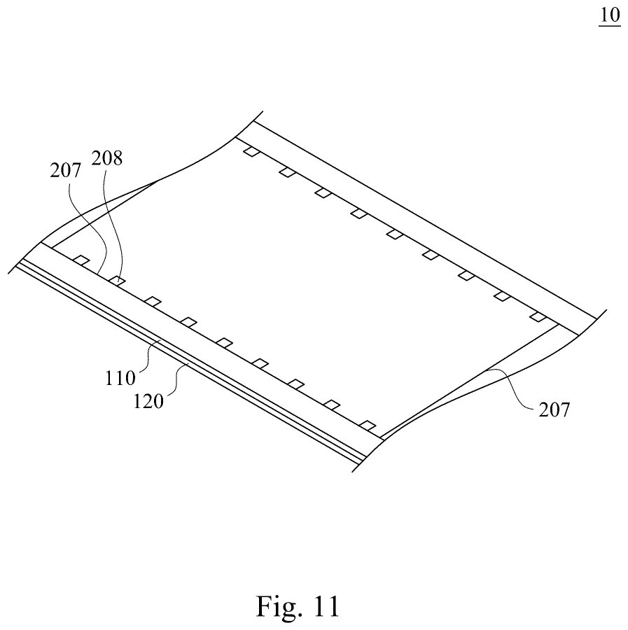

Please refer to FIG. 10, FIG. 10 shows a schematic view of medical images of patients printed on the bi-layer surgical guideline marking layer 10 in some embodiments of the present disclosure. In this example, the patient's medical image of the surgery is printed on the first layer 110 or under the second layer 120 of the bi-layer surgical guideline marking layer 10, or a combination thereof to form the medical image layer 206. In some examples, the medical image layer 206 includes, but is not limited to CT, MM, X-ray, ultrasound, thermal image and other auxiliary information to provide the information needed by the user during the surgery.

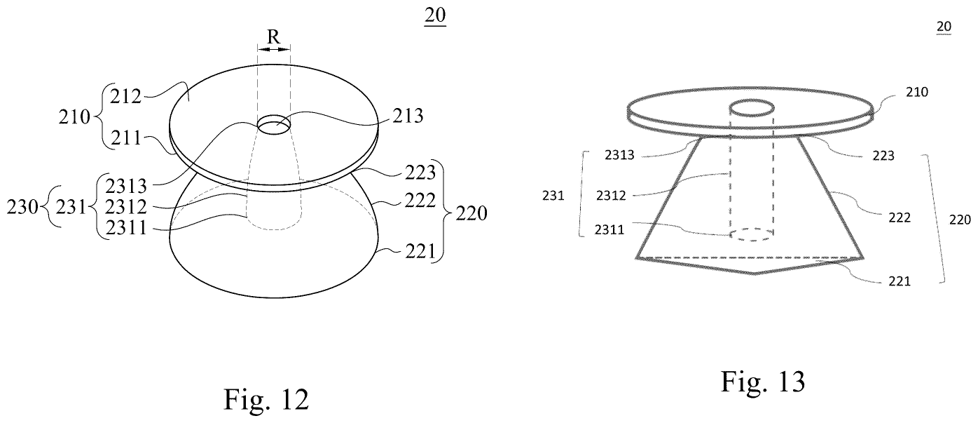

Please refer to FIG. 11, FIG. 11 shows a schematic view about a foldable wire 207 disposed around the bi-layer surgical guideline marking layer 10 or a signal element 208 disposed on the foldable wire 207 in some embodiments of the present disclosure. In some examples, the bi-layer surgical guideline marking layer 10 further includes a foldable wire 207, so that the bi-layer surgical guideline marking layer 10 can be better shaped, and then is fixed on the object surface. The foldable wire 207 uses soft glue to fix the foldable wire 207 around the first layer 110 and the second layer 120 to prevent the foldable wire 207 falling off when the bi-layer surgical guideline marking layer 10 is bent. Specifically, the foldable wire 207 is disposed between the first layer 110 and the second layer 120, and is around the first layer 110 and the second layer 120. In some other examples, the foldable wire 207 is disposed at opposite sides of the bi-layer surgical guideline marking layer 10. In some examples, the foldable wire 207 is disposed includes, but is not limited to on the first layer 110, under the first layer 110, on the second layer 120, under the second layer 120, or in the middle of the first layer 110 and the second layer 120. The foldable wire 207 is attached between the first layer 110 and the second layer 120 by using viscous adhesive. In some examples, the viscous adhesive is selected from materials that can still be bent together after bonding with the foldable wire 207. In some examples, the bi-layer surgical guideline marking layer 10 further includes at least one hole disposed on the foldable wire 207 to scrape the fluid in the receiving space to the first receiving tank 130, the second receiving tank 140, or a combination thereof; or the fluid is allowed to seep back into the receiving space (such as the first receiving tank 130 and/or the second receiving tank 140) from the receiving space. In some other examples, the bi-layer surgical guideline marking layer 10 further includes at least one signal element 208 disposed on the foldable wire 207. When the foldable wire 207 contacts to the surface of the patient, there will be a signal output or a color changing to determine whether the bi-layer surgical guideline marking layer 10 attaches the patient's body surface to achieve a better fixation effect.

The mode of a mono-layer surgical guideline marking layer.

In some other embodiments of this disclosure, a surgical guideline marking layer 1 (or surgical guideline mark structure) is provided that includes the mono-layer surgical guideline marking layer 11 and a fixing device 20, please refer to FIG. 12 to FIG. 24B. The mono-layer surgical guideline marking layer 11 is a transparent material, and its specific material can be the same as the bi-layer surgical guideline marking layer mentioned above, and those descriptions will not be repeated herein.

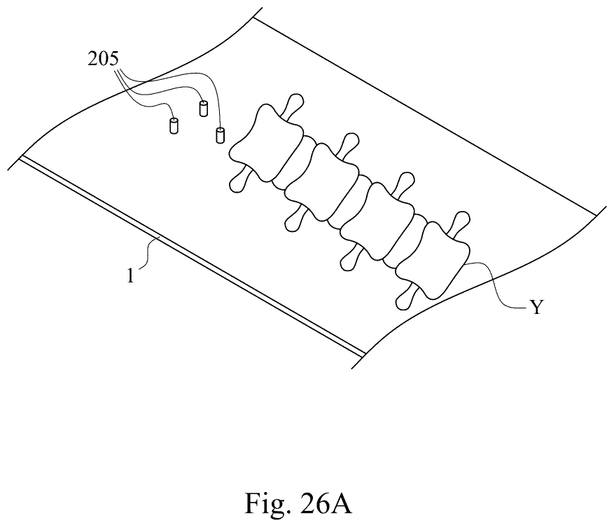

Please refer to FIG. 12, FIG. 12 shows a perspective view of a fixing device in some embodiments of the present disclosure. The fixing device 20 includes the parallel extended structure 210, the vacuum forming layer 220 and instrument guide structure 230. The parallel extended structure 210 is connected with the mono-layer surgical guideline marking layer 11 (as show in FIG. 23). The parallel extended structure 210 includes a first surface 211, a second surface 212, and a second instrument through hole 213 penetrating the first surface 211 and the second surface 212.

The vacuum forming layer 220 has a first end 221 of vacuum forming layer 220, a second end 223 of vacuum forming layer 220, and the connecting surface 222 between the first end 221 and the second end 223 of the vacuum forming layer 220. In some examples, the vacuum forming layer 220 is used to achieve vacuum forming adsorption. In some examples, the first end 221 may be circular.

The instrument guide structure 230 includes instrument guide layer 231 is disposed to connect the second end 223 of vacuum forming layer 220, and the second instrument through hole 213 of parallel extended structure 210. In some examples, the instrument guide layer 231 has a first end 2311 of the instrument guide layer 231, a second end 2313 of the instrument guide layer 231, and a connecting surface 2312 between the first end 2311 and the second end 2313 of the instrument guide layer 231; in which, the second end 223 of the vacuum forming layer 220 and the second end 2313 of the instrument guide layer 231 are connected with the second instrument through hole 213 of the parallel extended structure 210, in other words, the second instrument through hole 213 has the same inner diameter R as the instrument guide layer second end 2313. The instrument guide layer 231 can be used as a path guide for surgical instrument X.

In some examples, the inner diameter R on the second end 2313 of the instrument guide layer 231 and the size of the surgical instrument X are matched with each other, so the inner diameter R on the second end 2313 of the instrument guide layer 231 can be used as an auxiliary path guide at the same time. Surgeons can fix the position on the site to be operated on before the surgery, and perform the surgery through position guidance.

In some examples, the second end 2313 of the instrument guide layer 231 has an inner diameter R, and the external diameter of the surgical instrument X can be surrounded in the inner diameter R, so that the surgical instrument X can move within the instrument guide layer 231, that is external diameter of surgical instrument X can match with the second end 2313 inner diameter R of instrument guide layer 231.

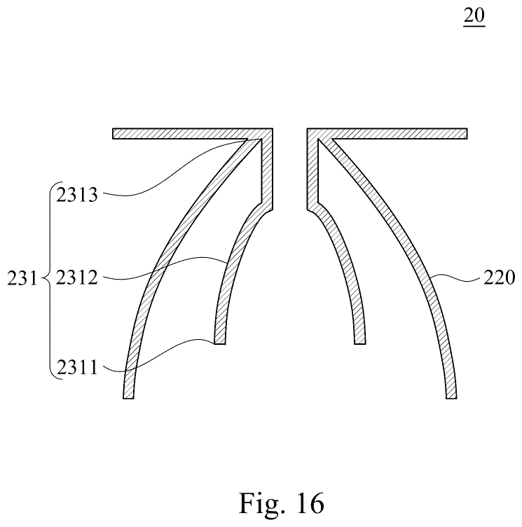

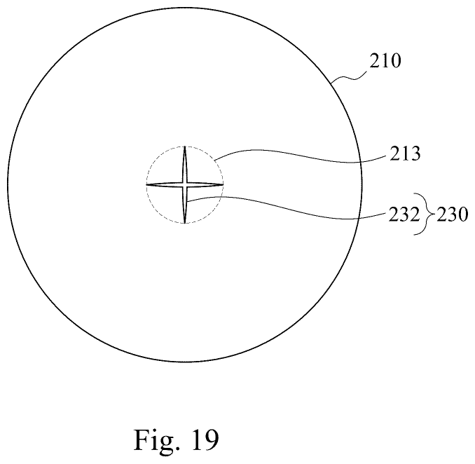

Please refer to FIG. 13 and FIG. 15 at the same time, FIG. 13 and FIG. 15 show three-dimensional schematic views of different examples of the fixing device 20 in some embodiments of the present disclosure. In some examples, the first end 221 of the vacuum forming layer 220 can be circular or polygonal, the connecting surface 222 between the first end 221 and the second end 223 of the vacuum forming layer 220 can be an arc surface, a plane or a combination thereof and the first end 2311 of the instrument guide layer 231 can be circular or polygonal, the connecting surface 2312 between the first end 2311 and the second end 2313 of the instrument guide layer 231 can be an arc surface, a plane or a combination thereof. The connecting surface 2312 or the first end 2311 can be designed in different shapes conform to the needs of the instruments used in different surgeries or the characteristics of the patient's body surface, so as to achieve the most suitable state for the surgery.

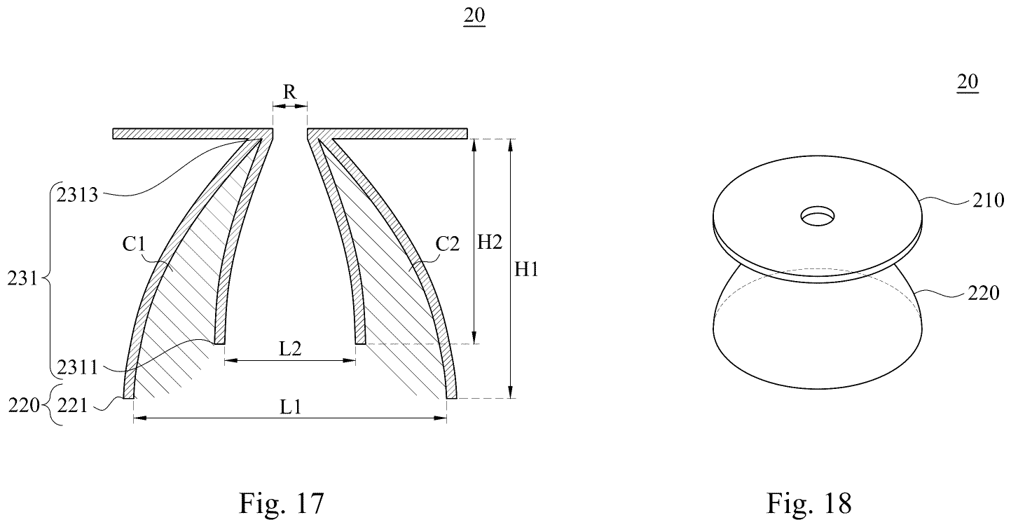

Please refer to FIG. 13 first, in some examples, the connecting surface 222 between the first end 221 and the second end 223 of the vacuum forming layer 220 is a plane, and the first end 221 of the vacuum forming layer 220 is a polygon; in some examples, the first end 221 can be circular (as shown in FIG. 12). The connecting surface 2312 between the first end 2311 and the second end 2313 of the instrument guide layer 231 is also a plane, and the first end 2311 of the instrument guide layer 231 is circular. Please refer to FIG. 14, FIG. 14 shows cross-sectional views of different examples of fixing devices in some examples of the present disclosure. In some examples, the connecting surface 2312 between the first end 2311 and the second end 2313 of the instrument guide layer 231 can be a plane as shown in a cross-sectional view.

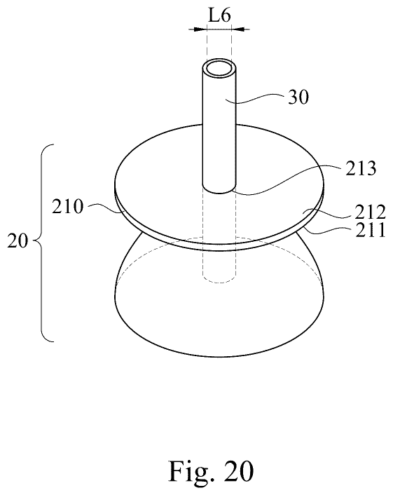

Please refer to FIG. 15, in some examples, the connecting surface 222 between the first end 221 and the second end 223 of the vacuum forming layer 220 is a plane, and the first end 221 of the vacuum forming layer 220 is circular. The connecting surface 2312 between the first end 2311 and the second end 2313 of the instrument guide layer 231 is a circular arc surface, and the first end 2311 of the instrument guide layer 231 is a polygon; in some examples, the first end 2311 may be circular (as shown in FIG. 13). Please refer to FIG. 16, FIG. 16 shows cross-sectional views of different examples of fixing devices in some examples of the present disclosure. In some examples, the connecting surface 2312 between the first end 2311 and the second end 2313 of the instrument guide layer 231 may also be a combination of a plane and an arc surface.

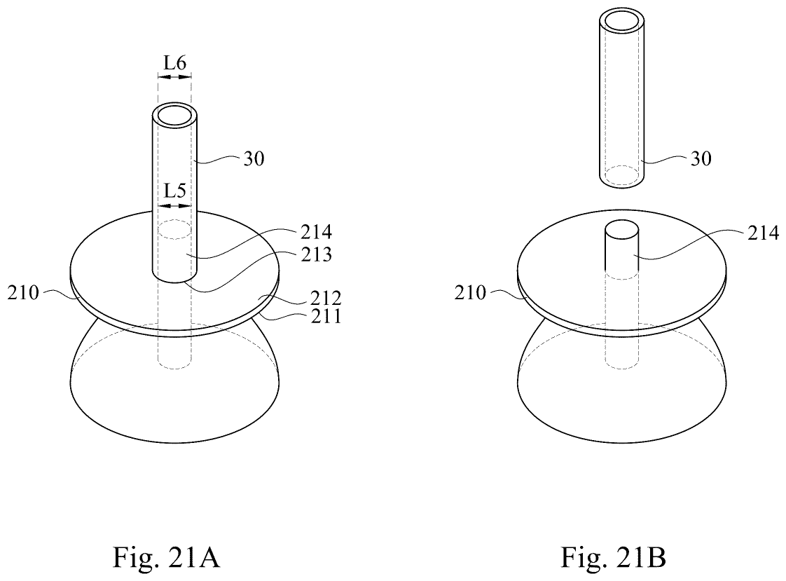

Please refer to FIG. 17, FIG. 17 shows three-dimensional section view of the fixing device 20. In some examples, the inner diameter L1 of the first end 221 of the vacuum forming layer 220 is greater than or equal to the inner diameter R of the second end 2313 of the instrument guide layer 231; and the inner diameter L2 of the first end 2311 of the instrument guide layer 231 is greater than or equal to the inner diameter R of the second end 2313 of the instrument guide layer 231 to ensure the degree of vacuum that can be fixed on the patient's body surface.

In some examples, the inner diameter L1 of vacuum forming layer first end 221 is related to vacuum suction, the larger the inner diameter L1, the stronger the suction. The inner diameter L1 of vacuum forming layer 220 first end 221 should be at least greater than or equal to the inner diameter R of the second end 2313 of the instrument guide layer 231. For example, when the inner diameter L1 of the vacuum forming layer 220 first end 221 is 1 cm, the suction force is about 800 grams (g); when the inner diameter L1 of the vacuum forming layer 220 first end 221 is 2 cm, the suction force is about 3 kilograms (kg). Similarly, the inner diameter L2 of the first end 2311 of the instrument guide layer 231 should be at least greater than or equal to the inner diameter R of the second end 2313 of the instrument guide layer 231.

In some examples, the difference between the inner diameter L1 of the first end 221 of vacuum forming layer 220 and the inner diameter L2 of the first end 2311 of the instrument guide layer 231 also affects the vacuum suction of the fixing device 20 at the same time, the greater difference between inner diameter L1 and inner diameter L2 is, the greater the suction force of the fixing device 20 will be. In addition, the greater the sum of volume C1 and C2 formed between the vacuum forming layer 220 and the instrument guide layer 231 is, the greater the suction force of the fixing device 20 will be.

In some examples, the height H2 of the instrument guide layer 231 does not exceed the height H1 of the vacuum forming layer 220 to ensure the full vacuum of the vacuum forming layer 220.

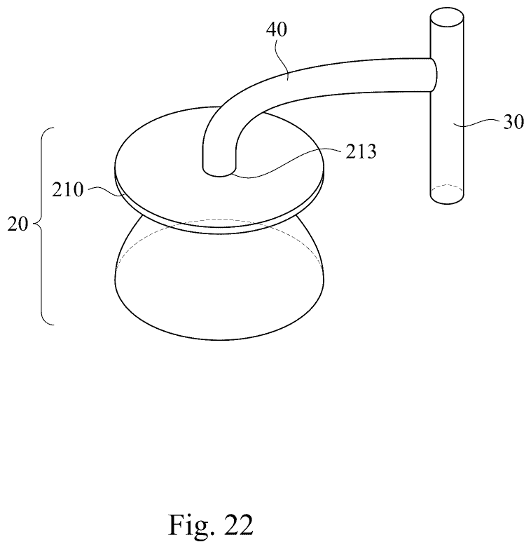

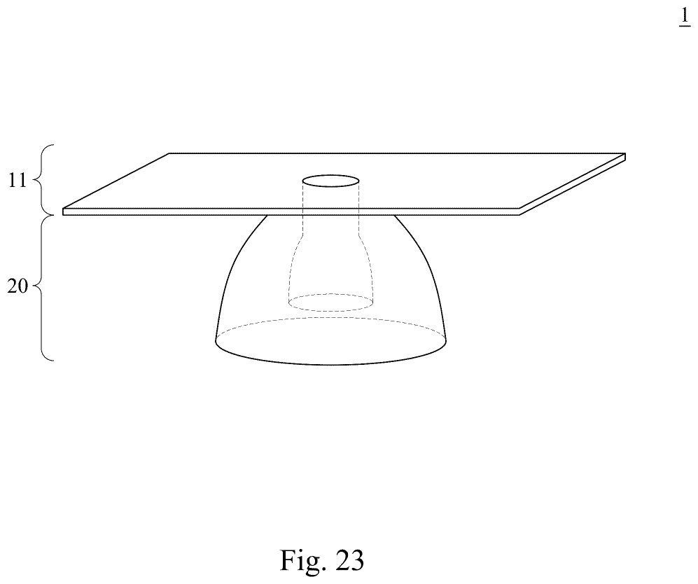

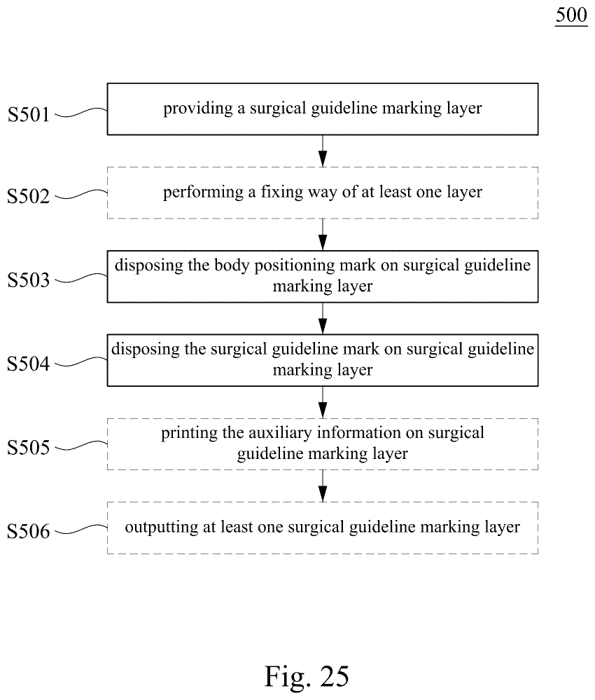

Please refer to FIG. 18, FIG. 18 shows a schematic illustration of an example without an instrument guide layer in a fixing device 20 in some embodiments of the present disclosure. In some examples, the shorter the height H2 of the instrument guide layer 231 is, the better the fixing effect of the fixing device 20 will be. The minimum range of the height H2 of the instrument guide layer 231 can be 0, and the maximum cannot exceed the height H1 of the vacuum forming layer 220. The example in FIG. 18 shows that the height H2 of the instrument guide layer 231 being 0 can be used as an examples that the instrument guide layer 231 is not required for this surgery. In some examples that the instrument guide layer 231 is not required, the fixing device 20 can be used in conjunction with other accessories to assist surgical guidance, such as valve 232 (as shown in FIG. 19) or surgical assistive device 30 (as shown in FIG. 22).

Please refer to FIG. 19, FIG. 19 shows a schematic top view of an embodiment of the second instrument through hole 213 on the parallel extended structure 210 of the fixing device 20 in some embodiments of the present disclosure. In some examples, the instrument guide structure 230 includes a valve 232, and the shape of the valve 232 includes, but is not limited to, a straight line, a cross, a hexagon, a pozi, or a star, etc., the valve 232 is disposed on the second instrument through hole 213 of the parallel extended structure 210. When the height H2 of the instrument guide layer 231 is 0 (as shown in FIG. 17 and FIG. 18), the second instrument through hole 213 on the parallel extended structure 210 can be designed to be adjustable in size. The second instrument through hole 213 can be composed of a valve 232, and the surgical instrument X can pass through the valve 232 to perform surgery under the valve 232. In this example, the combination of the parallel extended structure 210 and the valve 232 must enable the vacuum forming layer 220 to maintain a vacuum state and maintain the support of the surgical instrument X.

In some examples, fixing device 20 can be used together with surgical assistive device 30, for example: medical sleeves, dilators, etc. Surgical assistive device 30 should be hollow design, and can provide surgical instrument X a guiding path, so that surgical instrument X can pass through its hollow channel. The surgical instrument X, for example: a puncture needle, a guide wire, an endoscope, a syringe, etc. In some examples, fixing device 20 may be used in conjunction with surgical assistive device 30.

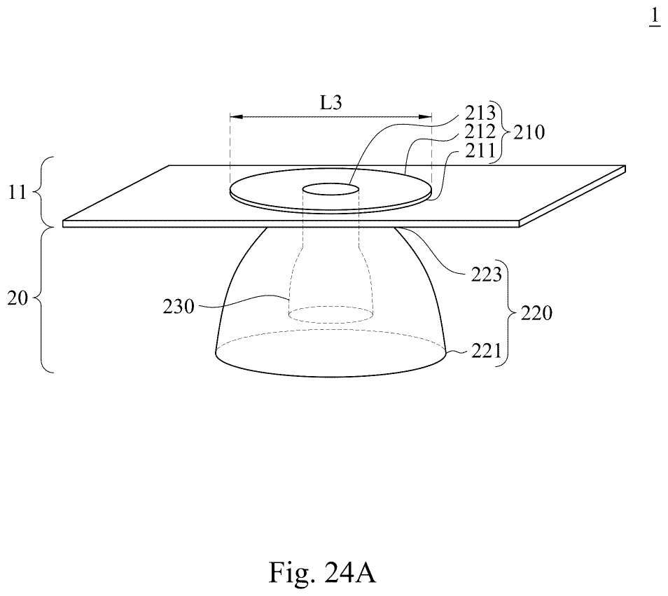

Please refer to FIG. 20, FIG. 20 shows the schematic about the fixing device 20 and the surgical assistive device 30 are integrally formed in some embodiments of the present disclosure. In some examples, the parallel extended structure 210 of the fixing device 20 further includes a transfer structure, in which the transfer structure includes: a surgical assistive device 30 in the form of a hollow barrel, and one end of the surgical assistive device 30 is disposed on the second instrument through hole 213. This example illustrates the integrated design of fixing device 20 and surgical assistive device 30. In this example, a surgical assistive device 30 integrally formed with the parallel extended structure 210 extends from the second surface 212 of the parallel extended structure 210 toward the direction away from the first surface 211 of the parallel extended structure 210, and the surgical assistive device 30 having an inner diameter L6 is equal to the diameter of the second instrument through hole 213 (that is, the instrument guide layer second end 2313 inner diameter R, as show in FIG. 17) on the parallel extended structure 210. Using such an integrated structure, the user doesn't need to waste the time to choose which surgical assistive device 30 to apply during the surgery.

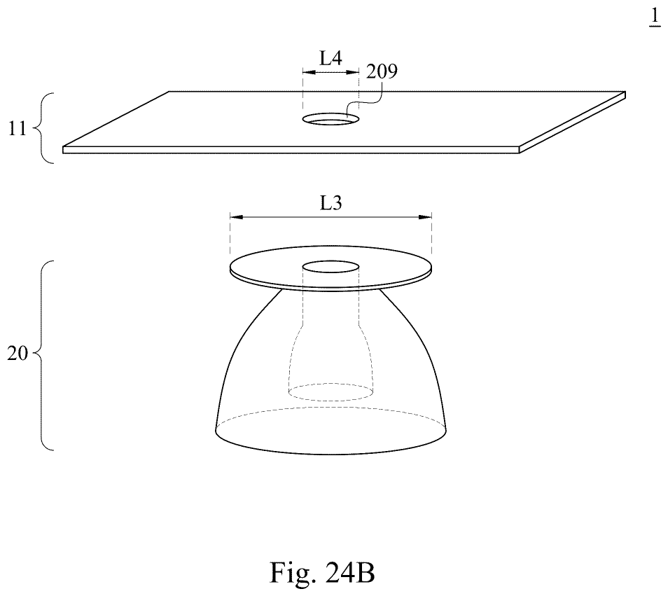

Please refer to FIG. 21A to FIG. 21B, FIG. 21A to FIG. 21B show a schematic view of the examples about combination and decomposition of the fixing device 20 and the surgical assistive device 30 in some embodiments of the present disclosure. In some examples, the parallel extended structure 210 of fixing device 20 includes a transfer structure, in which the transfer structure further includes an adapter 214 disposed on the second instrument through hole 213, and the adapter 214 is extended from the second surface 212 of the parallel extended structure 210 in a direction away from the first surface 211, and the surgical assistive device 30 is surrounded on the outer surface of the adapter 214.

In other words, the fixing device 20 and the surgical assistive device 30 can be of separate modular design. FIG. 21A is a schematic view of assembly, and FIG. 21B is a schematic view of disassembly. In this example, the second surface 212 of the parallel extended structure 210 can extend an adapter 214 toward the direction away from the first surface 211 of the parallel extended structure 210, and adapter 214 and parallel extended structure 210 are designed as integrally formed. The external diameter L5 of the adapter 214 and the inner diameter L6 of the surgical assistive device 30 can match and combine with each other, the combination can be in various forms, such as snap-in buckle, sticking, magnetic force, etc. With such a separate modular structure, several surgical assistive devices 30 can be replaced immediately during the surgery.

In some examples, the material of the surgical assistive device 30 can be designed according to the characteristics of the surgical instrument X that needs to be matched, for example: when the surgical instrument X is a puncture needle (steel needle, k-pin), a harder surgical assistive device 30 is used; when the surgical instrument X is a wire (Kirschner wire), a softer surgical assistive device 30 is used; and when the surgical instrument X diameter is different (that is: the instrument is not a cylinder), then a surgical assistive device 30 with a softer material can be used.

Please refer to FIG. 22, FIG. 22 shows a schematic view of fixing device 20, the flexible adapter 40 and the surgical assistive device 30 are used together in some embodiments of the present disclosure. In some examples, parallel extended structure 210 of fixing device 20 further includes: surgical assistive device 30 is a hollow barrel; and the flexible adapter 40. One end of the flexible adapter 40 is disposed on the second instrument through hole 213, and the other end of the flexible adapter 40 is disposed on the outer surface of the surgical assistive device 30. In other words, the flexible adapter 40 can be used as a connection between the fixing device 20 and the surgical assistive device 30, the connection between the flexible adapter 40 and the fixing device 20 or the surgical assistive device 30 can be in any way, for example: sticking, snap-in buckle, magnetic, or integrated.

The material of the flexible adapter 40 is a material with a flexible property, providing that the position and angle of the flexible adapter 40 are freely adjusted by the user during the surgery. The materials can be a single flexible material; or a combination of double materials, for example: the inner layer is made of aluminum wire, iron wire, copper wire or aluminum alloy and the outer layer thereof is coated with silicone.

The fixing device 20 can be used together with any product that needs to be fixed on the patient's body surface during the surgery, such as surgical drapes, surgical guideline patches, non-adhesive mono-layer surgical guideline marking layer 11, etc. The following paragraphs illustrate the example of mono-layer surgical guideline marking layer 11 with fixing device 20 (as shown in FIG. 23).

Please refer to FIG. 23, FIG. 23 shows a schematic view about the parallel extended structure 210 and the mono-layer surgical guideline marking layer 11 are integrally formed in the fixing device 20 in some embodiments of the present disclosure. In some examples, the parallel extended structure 210 and the mono-layer surgical guideline marking layer 11 are integrally formed, so that the first surface 211 of the parallel extended structure 210 is the lower surface of the mono-layer surgical guideline marking layer 11, and the second surface 212 is the upper surface of mono-layer surgical guideline marking layer 11 (as show in FIG. 12), that is, parallel extended structure 210 is the mono-layer surgical guideline marking layer 11. For example, the example of FIG. 23 illustrates the integrated form design of fixing device 20 and mono-layer surgical guideline marking layer 11, in this example, the fixing device 20 will be combined with the mono-layer surgical guideline marking layer 11 from the original parallel extended structure 210, and the integrally formed material can be silicone or TPU. Such an integrated design of the fixing device 20 and the mono-layer surgical guideline marking layer 11 is convenient for the user to directly read the information of the mono-layer surgical guideline marking layer 11 after fixing, and perform surgery immediately.

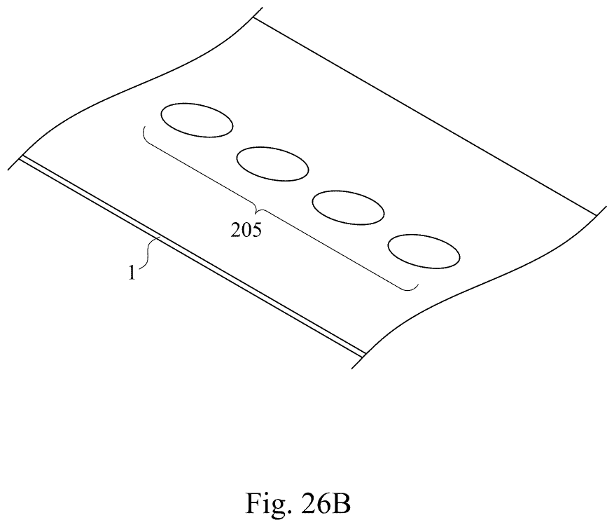

Please refer to FIG. 24A-24B, FIG. 24A-24B show a schematic view about the combination and decomposition of the fixing device 20 and the mono-layer surgical guideline marking layer 11 in some embodiments of the present disclosure. In some examples, the parallel extended structure 210 is fixed and connected (or integrally formed) with the vacuum forming layer 220, and the parallel extended structure 210 is passes through the opening 209 on the mono-layer surgical guideline marking layer 11 (for example, opening shapes such as a straight line, a cross, a hexagon, a pozi, a star or a ring, etc.). That is, fixing device 20 and mono-layer surgical guideline marking layer 11 can be a separate modular design, FIG. 24A shows an assembled schematic view, while FIG. 24B is an disassembled schematic view. In this example, the user must assemble the fixing device 20 with the pre-cut opening 209 on the mono-layer surgical guideline marking layer 11 before use, and the longest circumscribed circle diameter L4 of the pre-cut opening 209 on the mono-layer surgical guideline marking layer 11 must be smaller than the length L3 of the parallel extended structure 210 on the fixing device so that the assembly can be stable and will not easily fall or come out in other forms. The combination and disassembly design of this fixing device 20 and the mono-layer surgical guideline marking layer 11 is convenient for users to change multiple mono-layer surgical guideline marking layer 11 during the surgery.

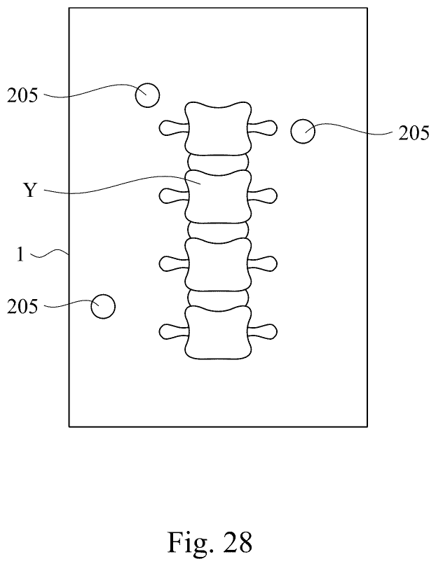

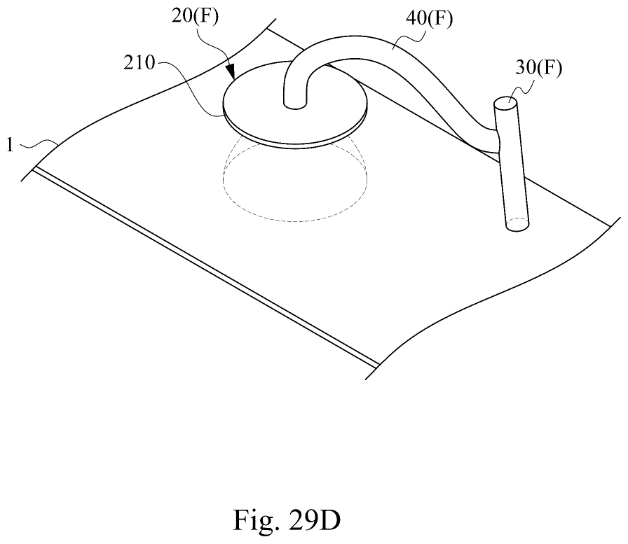

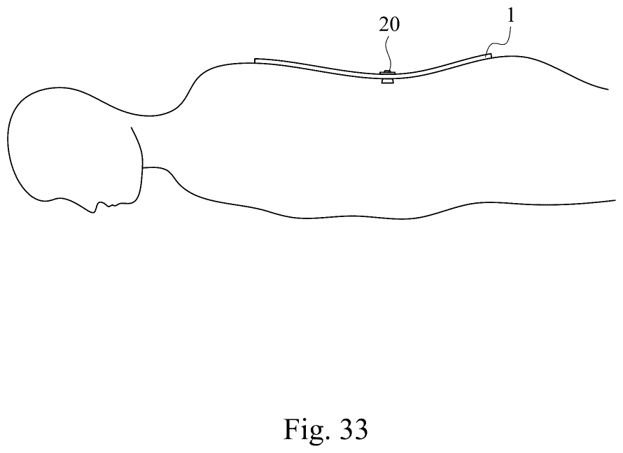

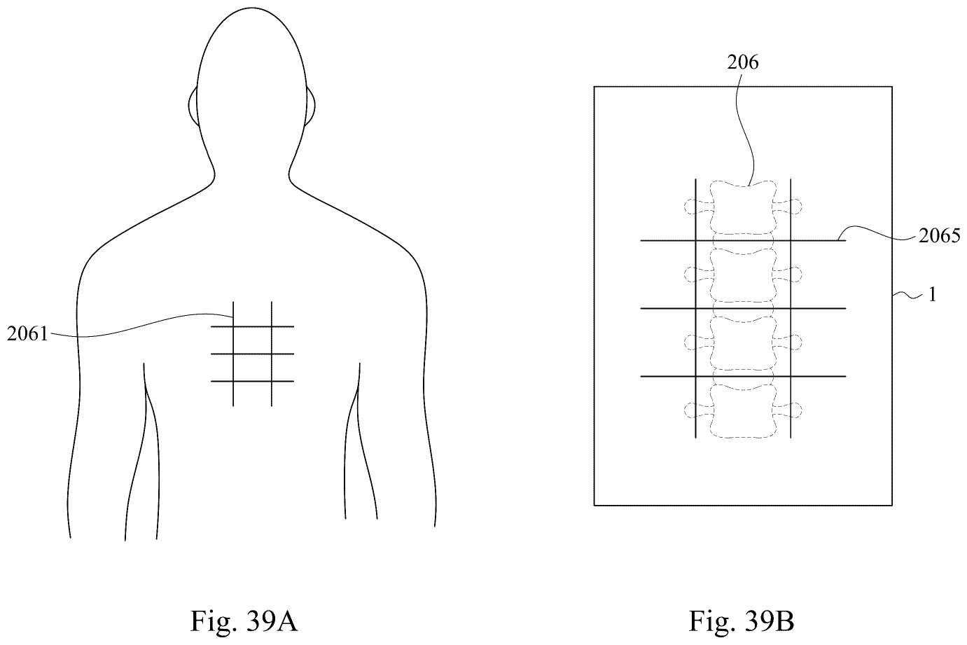

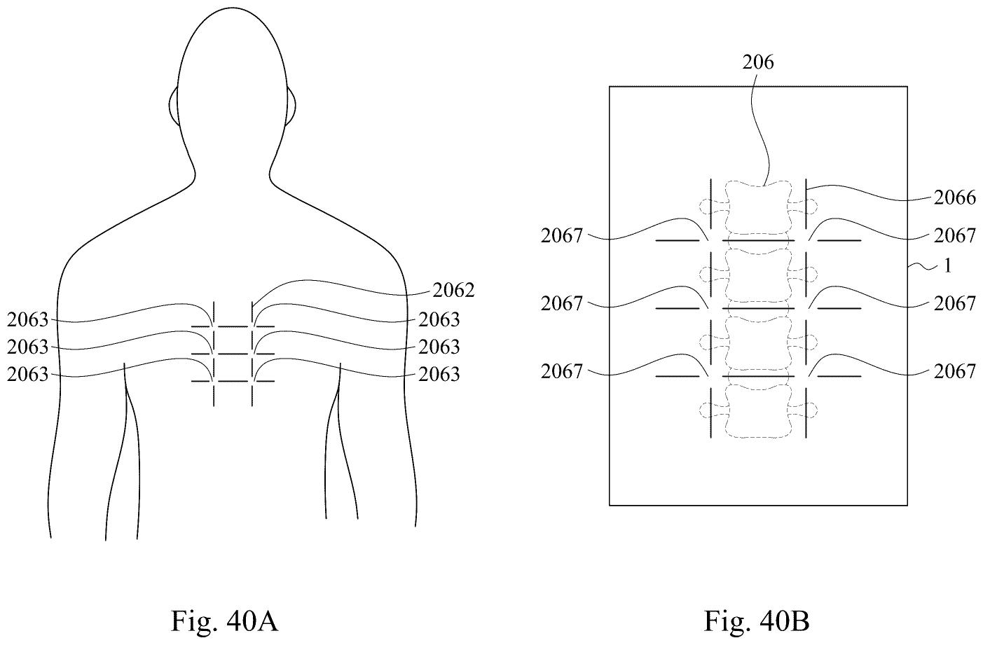

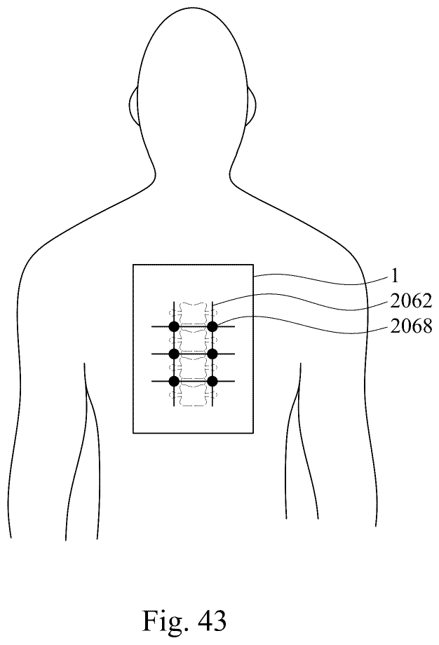

In some examples, surgical guideline marking layer 1 includes mono-layer surgical guideline marking layer 11 and body positioning mark 205. The body positioning mark 205 is an attaching device 201 (as shown in FIG. 29E), a surgical guideline mark F (as shown in FIG. 29A, FIG. 29B), a predetermined position 2052 (as shown in FIG. 34), object surface features, or called physiological characteristics Y (as shown in FIG. 26A, FIG. 29E), second continuous grid 2065 (as shown in FIG. 39A, FIG. 39B), a second discontinuous grid 2066 having at least one blank 2067 (as shown in FIG. 40A, FIG. 40B), at least one conducting blank mark 2068 relatively disposed at the at least one blank 2067 (as shown in FIG. 42), or a combination thereof.

In some examples, please refer backward to FIGS. 39A and 39B, body positioning mark 205 being the methods of production of second continuous grid 2065 includes disposing the first continuous grid 2061 (medical image imaginable mark) on the object surface, and reconstructing the curved surface of the object surface through the computer vision calculation. And then, the first continuous grid 2061 scans medical images together with the objects on the object surface to produce a surgical guideline marking layer 1 including the medical image layer 206 (that is, the object surface features, or physiological characteristics Y) and the second continuous grid 2065.

In some examples, please refer to FIGS. 40A and 40B. When the body positioning mark 205 is the second discontinuous grid 2066, the methods of production of second discontinuous grid 2066 includes disposing the first discontinuous grid 2062 (medical image imaginable mark) on the object surface, and reconstructing the curved surface of the object surface through the computer vision calculation. And then, the first discontinuous grid 2062 scans medical images together with the objects on the object surface to produce a surgical guideline marking layer 1 including the medical image layer 206 (that is, the object surface features, or physiological characteristics Y) and a second discontinuous grid 2066 having at least one blank 2067, at least one blank mark 2068, or a combination thereof.

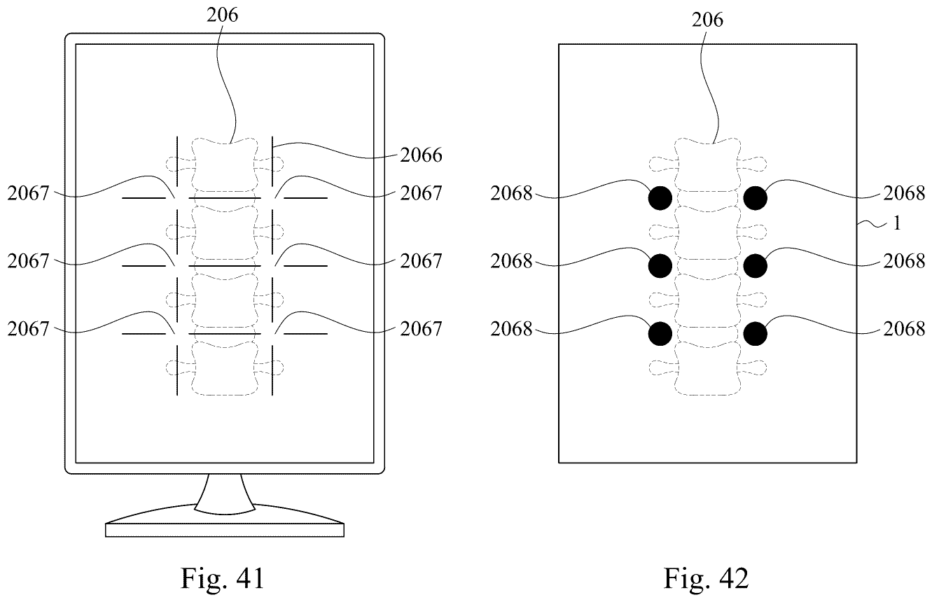

In some examples, please refer to FIG. 40A, FIG. 41 and FIG. 42, FIG. 41 and FIG. 42 are similar to FIG. 40A and FIG. 40B, the difference in FIG. 41 and FIG. 42 is the first discontinuous grid 2062 having at least one blank 2063 is a conductive material, and the surgical guideline marking layer 1 further includes at least one blank mark 2068, and at least one blank mark 2068 is disposed under the surgical guideline marking layer 1. In some examples, at least one blank mark 2068 is at least one conductive blank mark 2068.

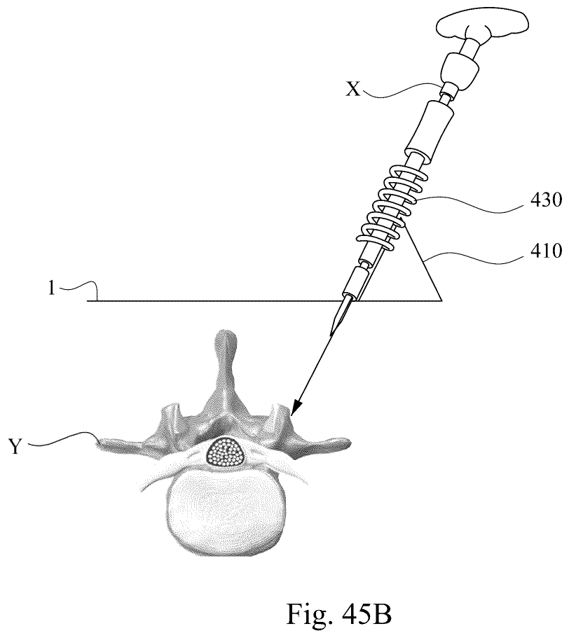

In some examples, please refer to FIGS. 45A and 45B, the surgical guideline marking layer 1 includes the mono-layer surgical guideline marking layer 11 and an auxiliary block 400 dispose on the mono-layer surgical guideline marking layer 11. In some examples, the auxiliary block 400 includes a main body 410 and a limit portion 420 disposed on top of the main body 410. In some examples, the auxiliary block 400 includes a main body 410 and a depth stopper 430 disposed on top of the main body 410.

The methods of producing of surgical guideline marking layer 1.

Although a series of operations or steps are used below to describe the method disclosed herein, an order of these operations or steps should not be construed as a limitation to the present invention. For example, some operations or steps may be performed in a different order and/or other steps may be performed at the same time. In addition, all shown operations, steps and/or features are not required to be executed to implement an embodiment of the present invention. In addition, each operation or step described herein may include a plurality of sub-steps or actions.

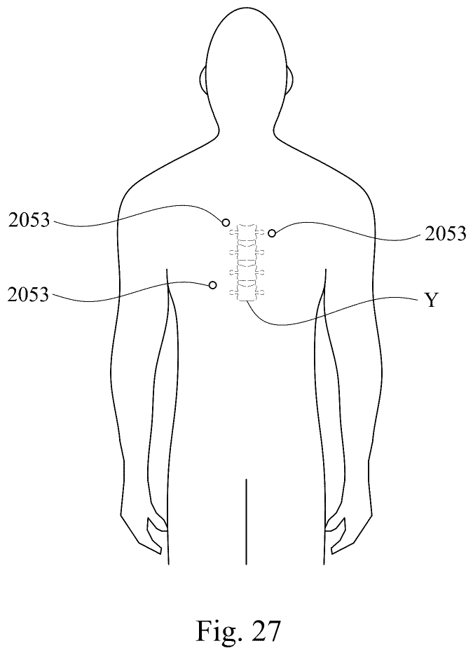

Please refer to FIG. 25, some embodiments of the present disclosure disclose a method of producing 500 of surgical guideline marking layer 1, the method of producing 500 of surgical guideline marking layer 1 includes the following steps:
Step S501, providing at least one layer;
Optionally entering step S502, and performing a fixing way of at least one layer;
Step S503, disposing the body positioning mark 205 on at least one layer, as a reference for disposing at least one layer on object surface;
Step S504, disposing the surgical guideline mark F on at least one layer as a guiding reference for the surgical path to obtain the surgical guideline marking layer 1;
Optionally entering to step S505, and printing auxiliary information on surgical guideline marking layer 1;
Optionally entering to step S506, and outputting at least one surgical guideline marking layer 1 according to surgical requirements. Specifically, the surgical guideline marking layer 1 here is the surgical guideline marking layer 1 with operation requirements.

In some embodiments, at least one layer includes, but is not limited to, the mono-layer layer or the bi-layer layer (for sample, the first layer 110 and the second layer 120 as shown in FIG. 1).

In some embodiments, the methods of producing 500 of surgical guideline marking layer 1 can make by producing the system of surgical guideline marking layer 1. The system of producing surgical guideline marking layer 1 (figure not shown) includes disposing device and marking device. The disposing device is configured to place the body positioning mark 205 on at least one layer, as a reference for disposing at least one layer of object surface features on the object surface. The marking device is configured to place the surgical guideline mark F on at least one layer, as a reference for the surgical path. In some examples, disposing devices include, but are not limited to inkjet printers, laser printers, 3D printers, UV printers, thermal transfer printers, laser cutters, wire cutters, laser engraving machine, bronzing machine, processing equipment, or robot arm, etc. In some examples, marking devices include, but are not limited to inkjet printers, laser printers, 3D printers, UV printers, thermal transfer machines, processing equipment, robotic arms, computer numerical control (CNC) engraving machines, lathes machine, milling machines, drilling machine etc. In some examples, when the disposing device and the marking device are used for printing, the materials that can be used include, but is not limited to common printing inks or printing materials that to be imaged in medical images, and the medical images include, but are not limited to: CT, MM, X-ray, ultrasound, thermal imaging, etc.

About step S502, in some embodiments, the step of performing the fixing way of the at least one layer, includes placing viscous adhesive under at least one layer, disposing the fixing device 20 (as shown in FIG. 12 to FIG. 24B) under at least one layer, disposing foldable wire 207 (as shown in FIG. 11) under at least one layer, or disposing at least one receiving tank (such as first receiving tank 130 in FIG. 1 and/or second receiving tank 140, for other detail modes, please refer to FIG. 1 above are all applicable, here wont′ description again) on the periphery of at least one layer.

About step S501 and S503, please refer to FIG. 26A to FIG. 26B, FIG. 26A to FIG. 26B shows a schematic view of surgical guideline marking layer 1 with body positioning mark. Please refer to step S501 and step S503 in FIG. 25 at the same time. After providing at least one layer (surgical guideline marking layer 1), body positioning mark 205 is a general printing ink or a material that to be imaged in medical imaging, and is printed on at least one layer (surgical guideline marking layer 1) of any position that does not block the surgical site. The body positioning mark 205 can be in any pattern, for example: square, circular, arbitrary polygon (as in FIG. 26A) or physiological characteristics Y (as in FIG. 26B, the pattern of body positioning mark 205 is the position of the spinous process of the spine), etc.

Please refer to FIG. 27, FIG. 27 shows a schematic view of using body positioning markers. In some examples, before the step of printed the body positioning mark 205 on at least one layer (surgical guideline marking layer 1) by a disposing device (such as a printer), the method includes placing the medical image imageable mark 2053 on the patient's body surface, and then scanning the medical image imageable mark 2053 having been placed on the patient's body surface by a scanning device. In some examples, the scanning devices include, but are not limited to computerized tomography scanners, X-ray machines, magnetic resonance imaging scanners, ultrasonic scanners, positron emission tomography, thermal imaging, etc.

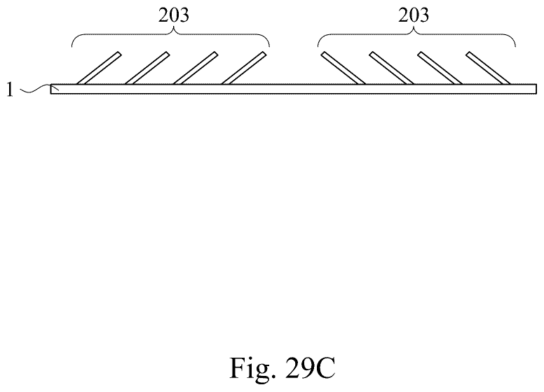

Please refer to FIG. 28, FIG. 28 shows a schematic view of the body positioning mark 205 in some embodiments producing in surgical guideline marking layer 1. In some examples, the user can output the image of the body positioning mark 205 together with other surgical information (such as the patient's physical characteristics Y) in an image, and the image can be printed on the surgical guideline marking layer 1 and can be printed on at least one layer (surgical guideline marking layer 1). The surgical information can include, but is not limited to: patient medical images, imageable markers, surgical guideline mark F, patient identification data, disease reminder, surgical reminder information, or one or any combination thereof.

Specifically, the medical image imageable mark 2053 can be fixed on the body surface of the patient when the patient scans the medical image, so that the medical image imageable mark 2053 can be scanned together with the patient. The image and other surgical information can be selectively printed on at least one layer (surgical guideline marking layer 1), and the body positioning mark 205 is produced on at least one layer (surgical guideline marking layer 1). And if the medical image imageable mark 2053 is necessary to be removed from the patient's body surface after scanning, and the position of the medical image imageable mark 2053 is necessary to be left on the patient's body surface. For example: a medical marking pen (marking pen) is used to mark the medical image imageable mark 2053, so that the body positioning mark 205 can be placed and aligned according to the recorded position. In other words, the medical image imageable mark 2053 is placed around the patient's physiological characteristics Y before scanning the medical image of the patient, and the medical image imageable mark 2053 should be notice to avoid the important anatomical features covered by the body positioning mark 205 in the medical image. In some examples, the medical image imageable mark 2053 on the body surface includes the following categories: (1) the material that to be imaged in medical imaging (for example: imageable adhesive tape) can be temporarily attached to the body surface for a period of time, so that the material that to be imaged does not need to be removed after the medical image is scanned; (2) the medical image imageable mark 2053 has a transferable feature, when placed on the body surface, the medical image imageable mark 2053 will automatically transfer the position mark to the body surface, instead of using a marker pen for marking; (3) the medical image imageable mark 2053 has the characteristics of creating indentations on the body surface. After removal, the indentation on the body surface can be used for alignment, or a marking pen can be used to deepen the mark according to the texture.

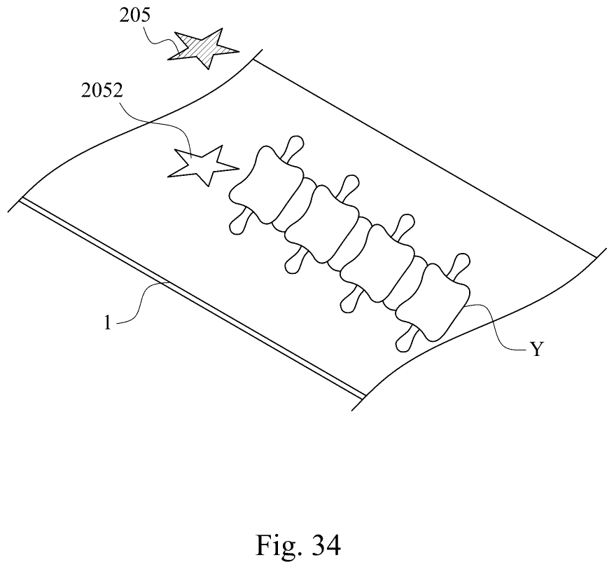

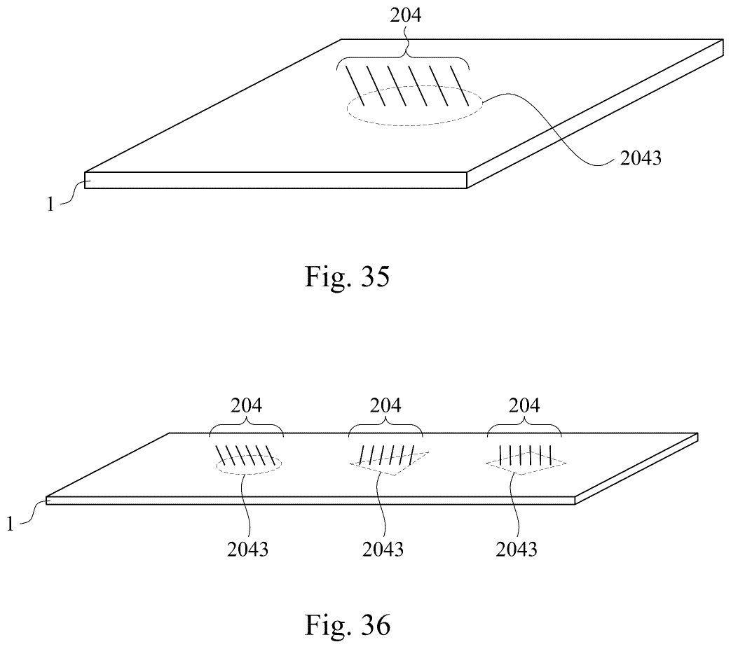

About step S504, please refer to FIG. 29A to FIG. 29B, FIG. 29A to FIG. 29B show a schematic view of surgical guideline marking layer 1 in the form of visual features in accordance with some embodiments of the present disclosure. Please also refer the step S504 in FIG. 25 at the same time, in some examples, the surgical guideline mark is disposed on at least one layer (surgical guideline marking layer 1) with a marking device. The surgical guideline mark F comprises a visual characteristic, a tactile characteristic, a physical object characteristic, or a combination thereof; in which the visual characteristic comprises circle, polygon, line, pinhead, fillet, surgical instrument copy shape F1, implant copy shape F2 (as shown in FIG. 29B), at least one grating structure 204 (as shown in FIG. 6, FIG. 35, or FIG. 36) with optical refraction, reflection, light concentrating effect, or a combination thereof. In some examples, after fixing the plastic sheet of the grating structure 204 by a marking device, a laser engraving machine is used to engrave a suitable grating size and shape on a plastic sheet. After fixing the plastic sheet, a printer is used to print out the grating shape; or after fixing the plastic sheet, the robotic arm grabs the glue and the robotic arm puts the glue on the position where the plastic sheet is to be pasted. And the robotic arm grabs the prepared grating sheet and pastes the prepared grating sheet on the plastic sheet. In some examples, visual images may be used as surgical guideline marks. As above mentioned, the surgical guideline marking layer 1 may include at least one type of surgical information or any combination thereof. FIG. 29A shows the combination images of surgical guideline mark F and patient physiological characteristics Y. In this example, the surgical guideline mark round end F01 can be used as the feature reference of the entry point of surgical instrument X on the patient's body surface, and the surgical guideline mark straight endpoint F02 is the endpoint of the target location of the surgery extended from the entry point of the patient's body surface, the surgical guideline mark straight endpoint F02 can be used as a reference for the path of instrument.

In some examples, the pattern of the surgical guideline mark is not limited to the pattern in FIG. 29A, for example: one end can be round, triangular, square; the other end can be pinhead, rounded, square, etc. Further, the surgical guideline mark F may also be the surgical instrument copy shape F1 expected to be used, or the implant copy shape F2 expected to be implanted in patient. Furthermore, the surgical guideline mark F can also include depth hint information. Please refer to FIG. 29B, this embodiment shows that the surgical guideline mark F on the surgical guideline marking layer 1 is the surgical instrument copy shape F1, and the surgical guideline mark F is the implant copy shape F2.

About step S505, please refer to FIG. 29A to FIG. 29B again, and please also refer to step S505 in FIG. 25. In some examples, further comprising printing auxiliary information on at least one layer (surgical guideline marking layer 1), in which the auxiliary information includes patient medical images, patient identification data, condition reminder, surgery reminder information, or a combination thereof. That is, FIG. 29A to FIG. 29B illustrate an embodiment that the auxiliary information is a medical image of a patient.

Please refer to FIG. 29C, FIG. 29C shows a schematic view of surgical guideline marking layer 1 in the form of tactile feature. The tactile feature includes at least one directional tactile structure 203, using the protruded structure in different directions, the user can feel the junction of the protruded structure with different directions by touching, and the junction is the reference position of the surgery path. In some examples, when the marking device is a robotic arm, after fixing the plastic sheet, the robotic arm grabs the glue and the robotic arm puts the glue on the plastic sheet. After putting the glue is completed, the robotic arm grabs the protruded structure (such as a ball, filament, or tentacles, in which the material of the protruded structure includes but is not limited to metal or plastic, etc.) and places the protruded structure on the position of putting glue.

Please refer to FIG. 29D, FIG. 29D shows a schematic view about physical object features of fixing device 20, surgical guideline marking layer 1, flexible adapter 40, and surgical assistive device 30 used together in some embodiments of the present disclosure, in which fixing device 20, surgical assistive device 30 and flexible adapter 40 can be used together as surgical guideline mark F. In FIG. 29D, the parallel extended structure 210 of fixing device 20 is located on the surgical guideline marking layer 1, and the dotted line is used to represent the structure of the fixing device 20 located below the surgical guideline marking layer 1. The devices shown in FIG. 29D include: fixing device 20, surgical assistive device 30, flexible adapter 40, or surgical guideline marking layer 1, all of them have different functions and have their own unique features. In some examples, all of the devices can be used alone or in combination with each other, users can choose any one or any combination according to their needs, or use all devices at the same time, for example: surgical assistive device 30 can be used with surgical guideline marking layer 1, surgical assistive device 30 and flexible adapter 40 can be used with surgical guideline marking layer 1, fixing device 20 can be used with surgical guideline marking layer 1, or any combination of fixing device 20, surgical assistive device 30, flexible adapter 40, surgical guideline marking layer 1, etc. can be used at the same time.

Please refer to FIG. 29E, FIG. 29E shows a schematic view of using attaching devices and hardware adapters at surgical guideline marking layer 1 in some embodiments of the present disclosure. The attaching device 201 may include body positioning mark 205, traceable devices, assembly accessories, etc. The trackable device may include, but is not limited to an optical tracking marker ball, a radio frequency (RF) device, etc. or a combination thereof.

About step S506, please refer back to FIG. 29A to FIG. 29E. In some examples, if multiple surgical guideline marks F is needs during the surgery, at least one surgical guideline marking layer 1 can be output. The step S506 as shown in FIG. 25, during the surgery, the user can place the surgical guideline marking layer 1 on the body surface of the expected operation site according to the body positioning mark 205 on the surgical guideline marking layer 1, the displayed methods of the body positioning mark 205 include: printing on at least one layer (surgical guideline marking layer 1), placing imageable markers on at least one layer (surgical guideline marking layer 1), scanning medical images with patients before surgery with imageable markers, or any one or any combination thereof. And no matter which displayed method is used, the use should be noted that the position of the body positioning mark 205 must avoid the actual part of the patient that needs surgery. In some examples, the user can select one or more the surgical guideline marking layer 1 to be output according to the patient's physical characteristics Y, the patient's body changes before and during the operation, or the complexity of the information on the surgical guideline marking layer 1. For example, if the medical image of the patient before the operation is scanned in a supine position, but the patient is in a prone position during the operation, the curvature of the spine will be slightly different. At this time, each vertebra can be independently output as a surgical guideline marking layer 1; or, when there are more than two types of information on surgical guideline marking layer 1, the information can also be output in separate sheets, so that one type of information is in one of the surgical guideline marking layer 1.

The methods of using surgical guideline marking layer.

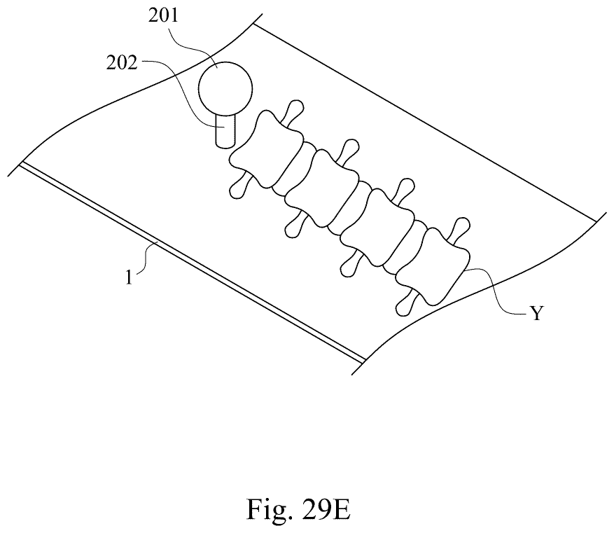

Please refer to FIG. 30, FIG. 30 illustrates the flow chart of using surgical guideline marking layer 1. First, in step S601, the methods of producing of the surgical guideline marking layer 1 are the descriptions in FIG. 25 of this disclosure and the corresponding paragraphs. After finish the methods of producing of surgical guideline marking layer 1, the following steps of use can be performed. The present disclosure also provides a methods of using 600 surgical guideline marking layer 1 including:
Step S601, providing at least one surgical guideline marking layer 1;
Step S602, fixing the surgical guideline marking layer 1 on the object surface;
Step S603, corresponding the identification mark or the object surface feature on the object surface to the body positioning mark 205 and/or the surgical guideline mark F to locate the surgical guideline marking layer 1;
Optionally entering step S604, and determining the attached degree of the surgical guideline marking layer 1 on the object surface.

About step S601, in some examples, at least one surgical guideline marking layer 1 includes, but is not limited to, mono-layer or bi-layer. In some examples, at least one surgical guideline marking layer 1 includes, but is not limited to 1. surgical guideline marking layer 1 having the body positioning mark 205 (as shown in FIGS. 26A and 26B); 2. surgical guideline marking layer 1 using attaching device 201 and hardware adapter 202 (as shown in FIG. 29E); 3. placing the medical image imageable mark 2053 on the patient's body surface first, and scanning together to produce body positioning mark 205 on the surgical guideline marking layer 1 (as shown in FIG. 27 and FIG. 28); 4. surgical guideline marking layer 1 having predetermined position 2052 and object surface features (as shown in FIG. 34); 5. the surgical guideline marking layer 1 having the second continuous grid 2065 (as shown in FIG. 39A and FIG. 39B), the second discontinuous grid 2066 having at least one blank 2067 (as shown in FIG. 40A and FIG. 40B), at least one conducting blank mark 2068 relatively disposed at the at least one blank 2067 (as shown in FIG. 42).

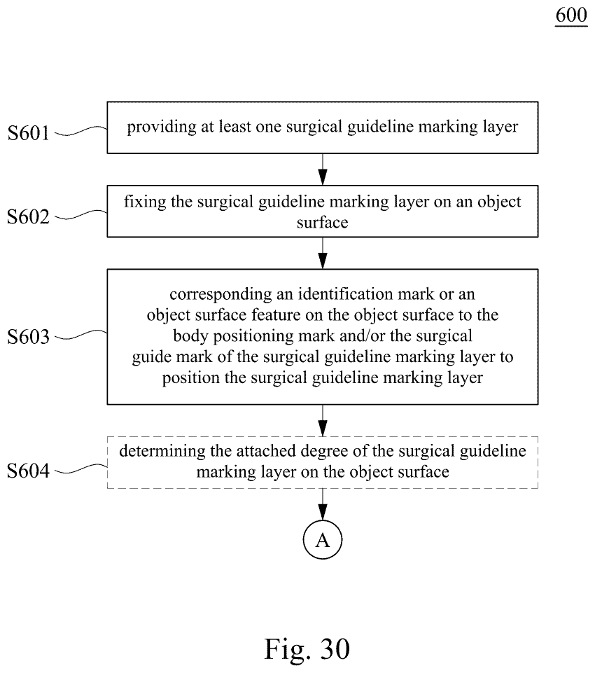

About step S602, in some examples, users can evaluate the appropriate fixation method on the patient's body surface according to the expected surgical site in the step of fixing the surgical guideline marking layer 1 on the object surface, FIG. 31 to FIG. 33 are schematic views of using different fixing ways to fix the surgical guideline marking layer 1 on the patient in some embodiments of the present disclosure.

Please refer to FIG. 31 first, in some examples, the friction or electrostatic attraction of the material of the surgical guideline marking layer 1 can be used to make the surgical guideline marking layer 1 adhere to the body surface of the surgery site. The surgical guideline marking layer 1 includes, but is not limited to, mono-layer or bi-layer, and can generate mono-layer surgical guideline marking layer 11 or bi-layer surgical guideline marking layer 10 according to user's needs, for example: when the surgical guideline marking layer 1 is required to repeatedly placed during the surgery, in order to avoid reducing friction, electrostatic attraction or stickiness, the bi-layer surgical guideline marking layer 10 can be used. The water between the first layer 110 and the second layer 120 of bi-layer surgical guideline marking layer 10 is scraped off to determine whether the first layer 110 and the second layer 120 are tightly bonded (as shown in FIG. 1). On the contrary, if the water is not easy to scrape off between the first layer 110 and the second layer 120 at the surgical site and the water is not easy to scrape off will be impossible to determine whether the first layer 110 and the second layer 120 are closely attached, the mono-layer surgical guideline marking layer 11 can be used (such as: at the temporomandibular joint or ear bone surgery).

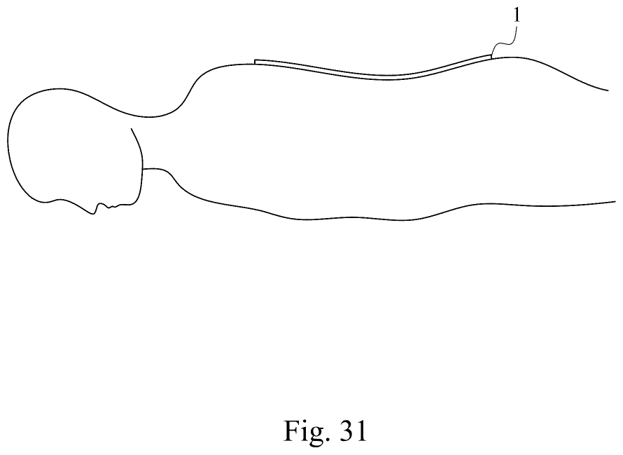

Please refer to FIG. 32, in some examples, the surgical guideline marking layer 1 with viscous adhesive G is used to fix the surgical guideline marking layer 1 on the body surface of the surgery site. For example, when the body surface of the surgical site is a smooth plane, the surgical guideline marking layer 1 can be used with viscous adhesive G or the electrostatic attraction of the surgical guideline marking layer 1 is used to fix the surgical guideline marking layer 1 on the patient's body surface.

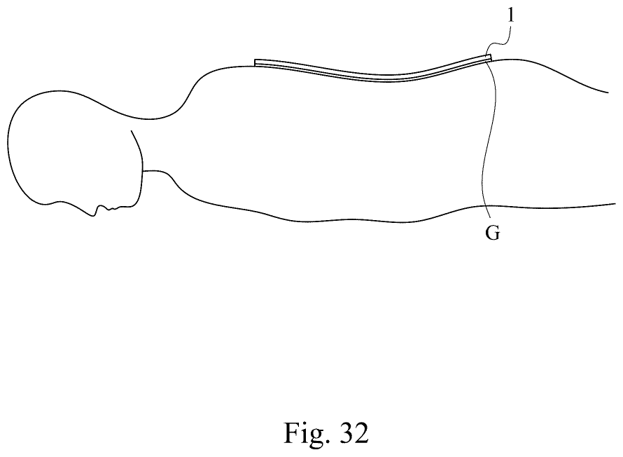

Please refer to FIG. 33, in some examples, the fixing device 20 can be further used with the surgical guideline marking layer 1, the fixing device 20 can be used with any product that needs to be fixed on the patient's body surface during the surgery, such as surgical drapes, surgical guideline patches, non-adhesive surgical guideline marking layer 1, etc. No matter which fixing way is used, the fixation method is necessary to ensure that the surgical guideline marking layer 1 is attached to the patient's body surface during the surgery, so as to avoid errors caused by the non-attach of the surgical guideline marking layer 1.

About step S603, in some examples, the body positioning mark 205 and/or the surgical guideline mark (the surgical guideline mark can also be used for body surface positioning) of the surgical guideline marking layer 1 correspond to the body surface features on the patient's body surface to place the surgical guideline marking layer 1. Please refer to FIG. 34, FIG. 34 shows a schematic view of pre-planning and placing body positioning mark predetermined position on the surgical guideline marking layer 1 in some embodiments of the present disclosure. Specifically, the predetermined position 2052 and object surface features (such as physiological characteristics Y) are printed on the surgical guideline marking layer 1, the object surface features on the surgical guideline marking layer 1 are corresponded to the object surface features on the object surface, and then the body positioning mark 205 is disposed on predetermined position 2052; the object surface is scanned to obtain medical images; and whether the distance between the body positioning mark 205 in the medical image and the surface feature of the object being the same as the distance between the body positioning mark 205 placed on the surgical guideline marking layer 1 and the surface feature of the object on the surgical guideline marking layer 1 is determined, if they are not the same, adjust the placement of surgical guideline marking layer 1 until they are the same.

In some other examples, in the step of aligning the surgical guideline marking layer 1 to the body surface, as shown in FIG. 26A and FIG. 29E, the surface features of the object (such as physiological characteristics Y) are printed on the surgical guideline marking layer 1, the surface features of the object printed on the surgical guideline marking layer 1 corresponds the object surface features on the surgical guideline marking layer 1 to the object surface features on the object surface.

In some other examples, in the step of aligning the surgical guideline marking layer 1 to the body surface, as shown in FIG. 27 and FIG. 28, after the medical image imageable mark 2053 on the object surface is imaged, the surgical guideline marking layer 1 forms body positioning mark 205; before the medical image imageable mark 2053 is removed, the position of the medical image imageable mark 2053 is marked on the object surface with the identification mark (such as marking with a marker pen); the identification mark on the object surface corresponds to the body positioning mark 205 of the surgical guideline marking layer 1 to locate the surgical guideline marking layer 1.

In some examples, a design for confirming whether the surgical guideline marking layer 1 is attached to the patient's body surface can be added. To confirm the attached degree of the surgical guideline marking layer 1 on the patient's body surface, the step S604 as shown in FIG. 30, the step S604 may be to add the known characteristics of the patient body curved surface information into the design of the surgical guideline marking layer 1 in any form. The patient body curved surface information includes position information and direction information. In addition, the patient body curved surface can be obtained by any methods, such as: direct measurement, calculation through markers disposed on the patient's body surface, calculation through image, computer vision calculation, etc.

About the step S604, please refer to the various embodiments in FIG. 35 to FIG. 43.

Please refer to FIG. 35. FIG. 35 shows a schematic view of a grating structure disposed at surgical guideline marking layer 1 in accordance with some embodiments of the present disclosure. In some examples, determining the attached degree of the surgical guideline marking layer 1 on the patient's surface includes using at least one grating structure 204 disposed on the surgical guideline marking layer 1, in which when the surgical guideline marking layer 1 is completely attached on the patient's body surface, the body positioning mark 205, surgical guideline mark F (as shown in FIG. 6), auxiliary information, or a combination thereof will be completely displayed by illuminated the grating structure 204 with light. Specifically, when the light irradiates the grating structure 204, the corresponding image information 2043 will be formed. In some examples, after obtaining the patient body curved surface information, the characteristics of the patient body curved surface information can be added to the surgical guideline marking layer 1 in the form of grating structure 204 and image information 2043, so as to confirm whether the surgical guideline marking layer 1 is in expectations of the patient's curved surface, that is, the surgical guideline marking layer 1 is located at the expected position and is attached to the curved surface of the patient's body. Further, the same surgical guideline marking layer 1 can have multiple groups of grating structure 204, and each group of grating structure 204 has its corresponding image information 2043, and the image information 2043 can be processed in surgical guideline marking layer 1 in any methods, the processing methods for example: printing or other processing methods.

Please refer to FIG. 36, FIG. 36 shows a schematic view of using at least one grating structure to determine the attached degree of the surgical guideline marking layer 1 in some embodiments of the present disclosure. At least one grating structure 204 is disposed on the surgical guideline marking layer 1. When the light illuminates the grating structure 204, the three grating structures 204 will form correspondingly different image information 2043. The three grating structures 204 are all designed to that when the surgical guideline marking layer 1 contacted by the grating structure 204 is attached to the expected curved surface of the patient's body, the grating structures 204 in the same group will uniformly face a specific angle. If user looks at surgical guideline marking layer 1 at a viewing angle parallel to the above specific angle, user can see the complete image information 2043 corresponding to grating structure 204.

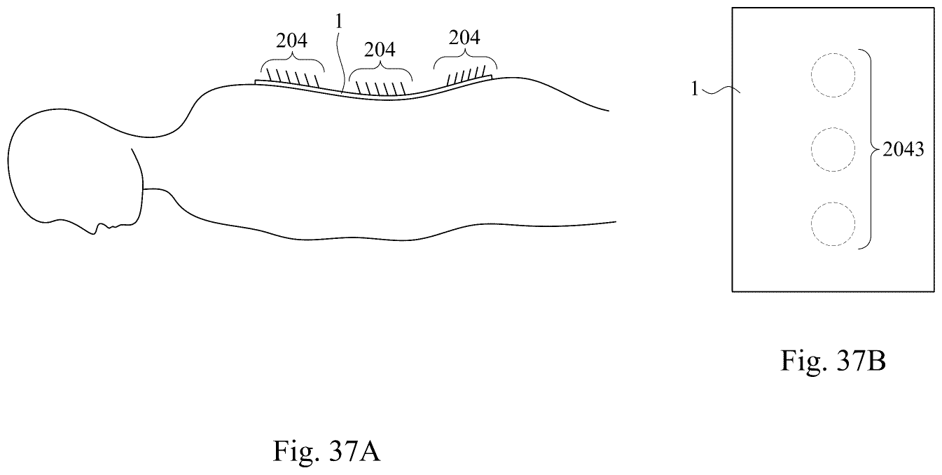

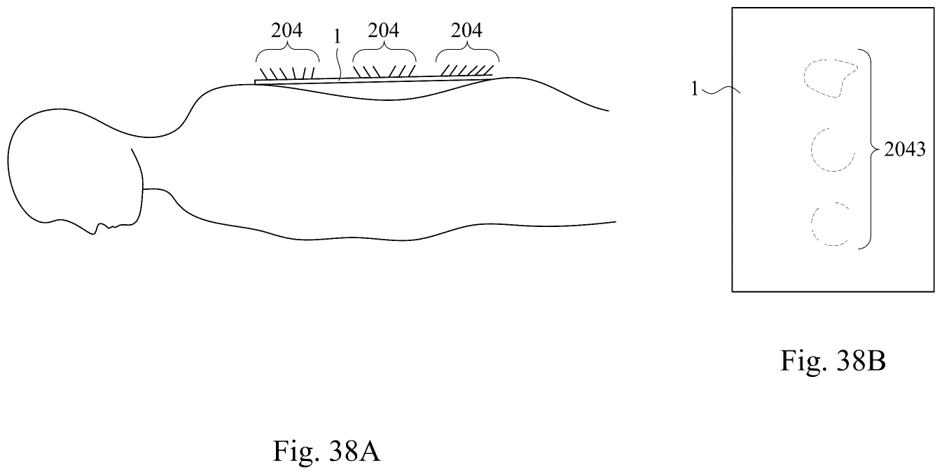

Please refer to FIG. 37A to FIG. 38B, FIG. 37A to FIG. 38B show the image information 2043 that the user can view when the same surgical guideline marking layer 1 is placed on the patient's body surface in different states. FIG. 37A to FIG. 37B show the state of the surgical guideline marking layer 1 attaching to the expected body surface of the patient; FIG. 38A to FIG. 38B show the state that the surgical guideline marking layer 1 is not attached to the expected curved surface of the patient's body.

Please refer to FIG. 37A first, FIG. 37A shows a schematic view of the surgical guideline marking layer 1 that has been completely attached to the surface of the patient in some embodiments of the present disclosure. When the surgical guideline marking layer 1 on the patient's body surface is located at the expected position on the patient's body surface and the surgical guideline marking layer 1 is attached to the patient body curved surface, the grating structure 204 groups on the surgical guideline marking layer 1 will all face specific directions. At this time, the user can view the complete image information 2043 corresponding to the grating structure 204 group through any grating structure 204 group, and the complete image information 2043 that the user can see is shown in FIG. 37B. FIG. 37B shows a schematic view of the complete image information in FIG. 37A. The image information 2043 corresponds to each one of the grating structures 204 respectively. It should be understood that the circular pattern is only used here as an illustration of the example, but the image information 2043 can be any shape, pattern, text, color block, etc.

Please refer to 38A again, FIG. 38A shows a schematic view of the surgical guideline marking layer 1 that has not been completely attached to the surface of the patient in some embodiments of the present disclosure. When the surgical guideline marking layer 1 on the patient's body surface does not conform to the curved surface of the patient's body surface, the grating structure 204 group will not be able to completely face their respective specific directions, resulting in the user being unable to view the complete image information 2043 through the grating structure 204 group. At this time, the image information 2043 viewed by the user is shown in FIG. 38B. FIG. 38B shows a schematic view of the not complete image information in FIG. 38A. The image information 2043 respectively corresponds to the grating structure 204 groups, and the image information 2043 is incomplete or deformed.

In some examples, the image information 2043 may include, but is not limited to: patient medical images such as: CT, MRI, X-ray, ultrasound, thermal images, etc., body positioning mark 205, surgical guideline mark F, patient identification, disease reminder, surgery reminder information, or one or any combination thereof.

Please refer to FIG. 39A and FIG. 39B at the same time, FIG. 39A and FIG. 39B show a schematic view of disposing the first continuous grid 2061 with the identifiable mark (such as a mark that can be recognized by a medical image, or called a medical image imageable mark) on the object surface. In some examples, the identifiable mark is disposed on the body surface of the patient before scanning medical images of the patient. The disposing method can be any method, for example: pasting, placing directly, or drawing on the patient's body surface with ink that to be imaged in medical images. The disposed pattern can also be in any form, for example: continuous grid, discontinuous line segment, text mark, physiological and anatomical feature mark and so on. Please refer to FIG. 39B, FIG. 39B shows the surgical guideline marking layer 1 produced when the first continuous grid 2061 with identifiable marks is disposed on the object surface (as shown in FIG. 39A) to take medical images together. In some examples, the curved surface of the patient's body surface can be reconstructed through computer vision calculation after the medical images are scanned in the form of grid marks.

Please also refer to FIG. 40A and FIG. 40B, FIG. 40A shows a schematic view of disposing the first discontinuous grid of identifiable mark on the object surface in some embodiments of the present disclosure. That is, the pattern of the identifiable mark can be designed as a partially hollow form. Among the identifiable marks on the patient's body surface, there are multiple disconnected first discontinuous grid blank 2063 between each line segment. The first discontinuous grid 2062 of the identifiable mark on the patient's body surface and the surgical guideline marking layer 1 second discontinuous grid 2066 (as shown in FIG. 40B) are connected and used together to provide the status information of the surgical guideline marking layer 1 placed on the patient's body surface for user.

In some examples, the step of determining the attached degree of surgical guideline marking layer 1 on the patient's surface includes: disposing identifiable marks that to be imaged in medical imaging on the patient's body surface, and reconstructing the curved surface of the patient's body surface through computer vision calculation, in which the identifiable marks contains first continuous grid 2061, first discontinuous grid 2062 having at least one blank 2063, or a combination thereof. Before the medical image is scanned by the patient, a mark that can be recognized by the medical image can be dispose on the patient's body surface. The medical images include, but are not limited to: CT, MRI, X-ray, ultrasound, thermal images, etc. After the patient has scanned the medical image, the medical image with identifiable marks can be extracted by computer vision calculation to extract the position of the mark and reconstruct the curved surface of the patient's body surface, the medical image with identifiable marks can be used as a benchmark for the curved surface of the patient's body surface. In some embodiments, the identifiable marks that to be imaged in medical imaging are disposed on the object surface. When placing the surgical guideline marking layer 1, the surgical guideline marking layer 1 can be placed according to the position disposed by the identifiable marks that to be imaged in medical imaging on the object surface of the object.

In some examples, the first discontinuous grid 2062 having identifiable marks is used to scan medical images together with the patient's body surface, and can produce a medical image layer 206, a second continuous grid 2065, a second discontinuous grid 2066 having at least one blank 2067, or a combination of thereof.

In some examples, the method for determining the attached degree on the patient's surface includes: disposing the surgical guideline marking layer 1 on the patient's body surface, in which, when the second continuous grid 2065 corresponds to the first continuous grid 2061, then the surgical guideline marking layer 1 is attached to the object surface; or when the second discontinuous grid 2066 having at least one blank 2067 corresponds to the first discontinuous grid 2062 having at least one blank 2063, the surgical guideline marking layer 1 is also attached to the object surface.

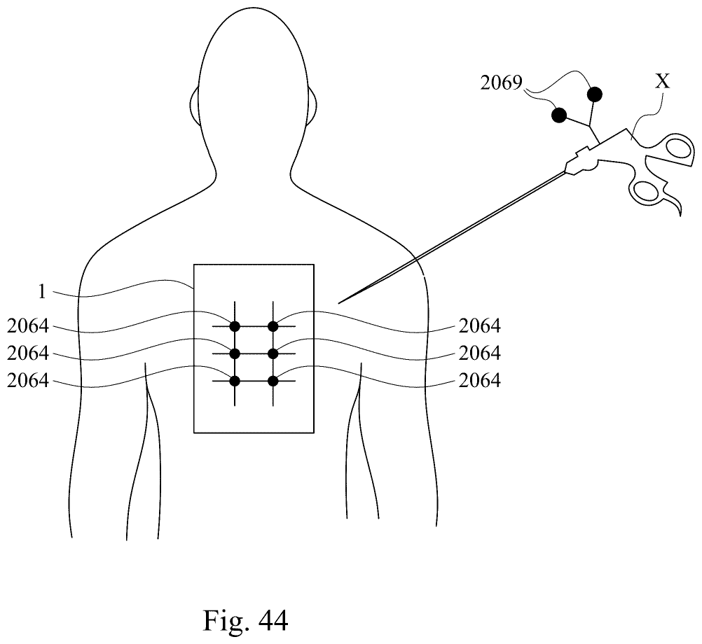

Please refer to FIG. 40A, FIG. 41, FIG. 42 and FIG. 43 at the same time. The identifiable mark is disposed on the object surface (FIG. 40A), and then the image is scanned. The conductive blank mark 2068 position extracted by blank 2067 through computer vision calculation is then produced in surgical guideline marking layer 1 (FIG. 42). The details are as follows.

As shown in FIG. 40A, first discontinuous grid 2062 (that is, medical image imageable mark) having multiple blank 2063 is disposed on the object surface.

As shown in FIG. 41, there are identifiable marks and medical images of patients in the computer images at the same time of some embodiments disclosed in the present disclosure. After implementing the step in FIG. 40A, the first discontinuous grid 2062 and the object on the object surface will scan medical images together. The medical image after scanning will have both the medical image layer 206 of the patient and the second discontinuous grid 2066 image produced by the first discontinuous grid 2062 and blank 2067. According to above mentioned, the surgical guideline marking layer 1 can use processing methods to make its surface having surgical information required in any surgery. Therefore, in this example, either or both of the medical image layer 206 of the patient and the image of the identifiable marker can be processed in the surgical guideline marking layer 1 at the same time.

Furthermore, in some examples, the curved surface of the patient's body surface can also be obtained through computer vision calculation and the relative information of the blank 2067 of the identifiable mark can be extracted, including but not limited to: position information, distance information of each blank 2067, shape information, size information, etc. and process the relative information features of the blank 2067 to surgical guideline marking layer 1. The range from the blank corresponding to the grid to other blanks around the blank can be regarded as attached (for example, the blank corresponding to one of the grids is a center of the circle, and the distance between the blank and other blanks is a radius, and the area drawn by the center of the circle and the radius is regarded as attached. As for whether other blanks are determined to be attached, the determination is also necessary to determine one by one based on this principle); the interval of blank can be designed according to the surface curvature of the object, for example, when the surface curvature of the object is smaller (smooth), the blank interval is larger; the surface curvature of the object is larger (steeper), the blank interval is smaller.

Please refer to FIG. 42, FIG. 42 shows a schematic view of the corresponding relationship between the patient medical image layer 206 and the blank mark 2068 of surgical guideline marking layer 1. After implementing the step in FIG. 41, the surgical guideline marking layer 1 with the patient's medical image layer 206 and at least one blank mark 2068 is generated. A plurality of blank mark 2068 corresponds to a plurality of blank 2067 images of the second discontinuous grid 2066 in FIG. 41. In other words, the features of blank mark 2068 on surgical guideline marking layer 1 include: position, size, shape, distance, etc. are designed based on the data of computer vision calculation.

Please refer to FIG. 43, FIG. 43 shows a schematic view of the connection of the surgical guideline marking layer 1 disposed on the body surface in some embodiments of the present disclosure. After implementing the step in FIG. 42, then, disposing the surgical guideline marking layer 1 on the body surface, and when at least one blank mark 2068 corresponding to at least one blank 2063 of the first discontinuous grid 2062 on the body surface is determined that the surgical guideline marking layer 1 is attached to the object surface.

For example, the first discontinuous grid 2062 of the identifiable mark on the patient's body surface is designed as a conductive substance, and the blank mark 2068 of the second discontinuous grid 2066 on the surgical guideline marking layer 1 is designed to have a conductive circuit. At this time, electric current is applied to each mark on the surgical guideline marking layer 1 in turn, that can help the user to identify whether the placement position of the surgical guideline marking layer 1 is consistent with the mentioned above curved surface of the computer vision calculation, and whether surgical guideline marking layer 1 is attached to the patient's body surface. In other words, if a path is generated, the path means that the surgical guideline marking layer 1 is attached to the patient's body surface and the position is correct; while a broken circuit means that the surgical guideline marking layer 1 is not attached to the patient's body surface or the position is deviated.

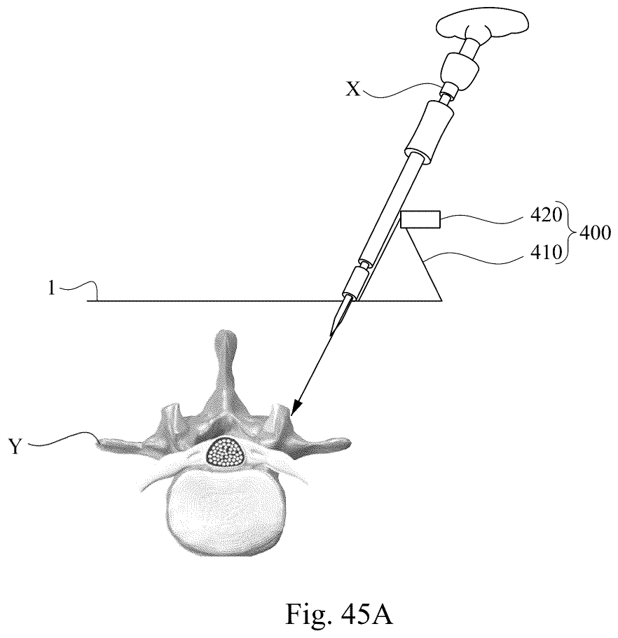

Please refer to FIG. 44. In some examples, multiple conductive blank marks 2068 of the surgical guideline marking layer 1 can be designed to have first distance sensors 2064, and can be measured distance between each first distance sensor 2064 through the first distance sensors 2064. By comparing the distance measured by each first distance sensor 2064, the distance between each blank 2067 is extracted through the computer vision calculation mentioned above (as shown in FIG. 41). The user can identify the position where the surgical guideline marking layer 1 is placed, and the user can determine whether the surgical guideline marking layer 1 is attached to the surface of the mentioned above computer vision calculation and the surgical guideline marking layer 1 is attached to the patient's body surface.

Please refer to FIG. 44 again, FIG. 44 shows a schematic view of a triangulation measurement that can be performed with the first distance sensor 2064 and the second distance sensor 2069 on the surgical instrument X in conjunction with the surgical guideline marking layer 1 in accordance with some embodiments of the present disclosure. In some examples, in addition to designing the marking on the surgical guideline marking layer 1 to have the first distance sensor 2064, one or more second distance sensors 2069 can also be added to the surgical instrument X. Triangulation measurement is measured by using any first distance sensor 2064 on surgical guideline marking layer 1 corresponding to the second distance sensor 2069 on surgical instrument X, the relative relationship between the current patient and surgical instrument X can be known.

Furthermore, if the user performs surgical planning through medical images in any surgical planning system software, the surgical instrument X and surgical guideline can be stored in the surgical planning system software. When the surgical instrument X reaches the user's expected surgical path, the expected relative relationship between the surgical instrument X and the sensors on the surgical guideline marking layer 1 can be stored in the surgical planning system software, and the user can compare the expected relative relationship with the current relative relationship of the sensors on surgical instrument X and surgical guideline marking layer 1 in a timely during the surgery. When the relative relationship between the surgical instrument X and the sensors on the surgical guideline marking layer 1 is inconsistent, that means the current surgical instrument X has not reached the expected surgical path. At this time, one or more warning methods can be used to remind the user, including but not limited to: visual, auditory, tactile prompts, etc.; when the relative relationship between the surgical instrument X and the sensors on the surgical guideline marking layer 1 is consistent, that means the current surgical instrument X has reached the expected planning path position, and then achieves the effect of surgical navigation.

Please refer to FIG. 45A, FIG. 45A shows a schematic view of using the auxiliary block 400 together with the surgical guideline marking layer 1 to assist the surgical instrument X in some embodiments of the present disclosure. In some examples, surgical guideline marking layer 1 includes the mono-layer surgical guideline marking layer 11 and the auxiliary block 400, the auxiliary block 400 further includes main body 410 and limit portion 420. The bottom of the main body 410 is disposed on the mono-layer surgical guideline marking layer 11, and the limit portion 420 is disposed on the top of the main body 410. In some examples, the way of disposing the bottom of the main body 410 on the mono-layer surgical guideline marking layer 11 includes, but is not limited to sticking or magnetic attraction (such as the mono-layer surgical guideline marking layer 11 having a magnetic substance, and the bottom of the main body 410 also having a magnetic substance, so that they can be attracted to each other), etc. Specifically, surgical guideline marking layer 1 can be used with auxiliary block 400 to prevent surgical instrument X from slipping or prevent the operation from being too deep when the user is operating on non-complete plane or difficult surgeries. For example, the main body 410 of the auxiliary block 400 can be used to prevent the surgical instrument X from slipping when the user performs operations on bone bevel such as the spinous process, ilium, or pedicle, etc.; or, for example, when the surgical site is close to important nerves or blood vessels, the limit portion 420 of the auxiliary block 400 can be used to further limit the depth of the surgical instrument X entering the body surface to prevent damage to the nerves or blood vessels.

Please refer to FIG. 45B, FIG. 45B shows a schematic view of using the auxiliary block 400 together with the surgical guideline marking layer 1 to assist the surgical instrument in another embodiments of the present disclosure. In some examples, the surgical guideline marking layer 1 includes the mono-layer surgical guideline marking layer 11 and the auxiliary block 400, and the auxiliary block 400 includes the main body 410 and the depth stopper 430. The bottom of the main body 410 is disposed on the mono-layer surgical guideline marking layer 11, and the depth stopper 430 is disposed on the top of the main body 410. Specifically, the depth stopper 430 includes, but is not limited to, compressed springs. For example, when the surgical site is close to the target site (such as a nerve or blood vessel), the surgical instrument X starts to touch the top of the compressed spring; when the surgical instrument X is necessary to get closer to the target position, the reaction resistance generated by the compressed spring starts to compress, so that the speed of the surgical instrument X approaching the target position will be slowed down, and the reaction resistance can even further limit the specific depth of pushing down to prevent the target position from being damaged.

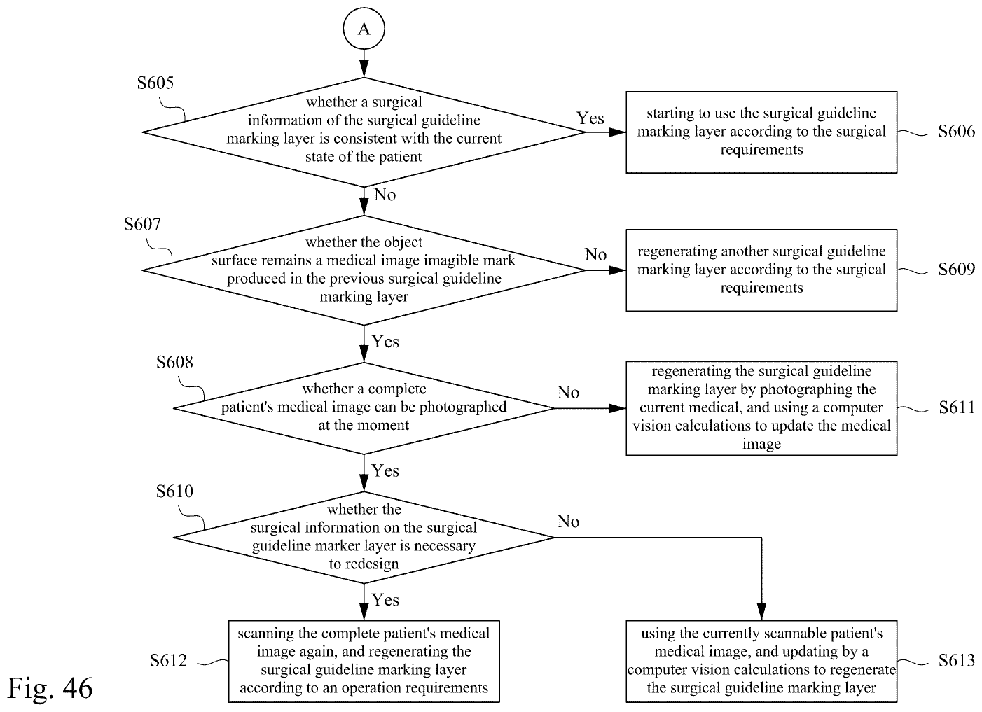

Please refer to FIG. 46, FIG. 46 is a flow chart of a method of using 600 surgical guideline marking layer 1 following in FIG. 30 according to some embodiments of the present disclosure. The present disclosure provides the methods of using 600 surgical guideline marking layer 1 further includes: determining whether the surgical information on the surgical guideline marking layer 1 is consistent with the current state of the patient. If so, start using the surgical guideline marking layer 1. Specifically, in step S605, the user can determine whether the surgical guideline marking layer 1 is consistent with the current state of the patient. Here should be understood that during the process of producing the surgical guideline marking layer 1, the information displayed on the surgical guideline marking layer 1 is all produced for the current medical image scan including: the medical image information of the patient, the surgical guideline mark F, the body positioning mark 205, patient body curved surface obtained through computer vision calculation, etc. However, in clinical, the state of the patient at the time of the surgery may be slightly different from the previous medical imaging due to any reason, for example: the difference between the scanning of the medical image and the current operation, the difference in the posture of the patient, the weight, etc. Therefore, the user must reconfirm whether the obtained surgical guideline marking layer 1 is conformed to the current state of the patient during the use process. If the surgical guideline marking layer 1 is attached to the current state is confirmed, then go to step S606, and the user can start using the surgical guideline marking layer 1 according to the surgical requirements.

In some examples, in step S605, determining whether the surgical information on the surgical guideline marking layer 1 is consistent with the current state of the patient. If not, go to step S607, determining whether the object surface remains a medical image imageable mark produced in the previous surgical guideline marking layer 1 (such as the first continuous grid 2061 or the first discontinuous grid 2062). If not, go to step S609, regenerating another surgical guideline marking layer 1 according to the surgical requirements. Specifically, if the current state is not consistent, that user is necessary to proceed to step S607. At this time, the user must determine again whether there is an identifiable mark left on the patient's body surface when the surgical guideline marking layer 1 was produced last time. If there is no identifiable mark on the patient's body surface when the surgical guideline marking layer 1 was produced last time, or the mark has been deformed, worn, missing, etc., the user needs to regenerate the surgical guideline marking layer 1 according to the surgical requirements. The method of producing is also described in FIG. 25 and FIG. 25's corresponding paragraphs.

In some examples, in the step S607, determining whether the object surface remains a medical image imageable mark produced in the previous surgical guideline marking layer 1 (such as the first continuous grid 2061 or the first discontinuous grid 2062). If so, go to step S608 to determine whether the complete medical image of the patient can be scanned at the moment, if not, go to step S611, regenerating the surgical guideline marking layer 1 by scanning the current medical image of the patient, and using a computer vision calculations to update the medical image of the patient. Specifically, if the currently available medical images cannot have complete patient information, the user can obtain the information of the identifiable marks on the patient's body surface through the currently available images, and the information includes position information and angle direction Information. After obtaining the image, the current patient body curved surface can also be obtained by computer vision calculation. At this time, the computer can further use the patient surface obtained through computer vision calculation during the previous production of surgical guideline marking layer 1. The patient body curved surface is used as a reference, and the change of the patient body curved surface can be obtained by comparing the two times patient body curved surface information and finding the conversion relationship between the identifiable marks in the two sets of images. And furthermore, the surgical information on the surgical guideline marking layer 1 is updated through the computer. In some examples, the images that can be scanned at the moment may be medical images or other visible light photography.

In some examples, in the step S608, determining whether a complete patient's medical image can be scanned at the moment, if so, go to step S610, determining whether the surgical information on the surgical guideline marker layer 1 is necessary to redesign, if so, go to step S612, scanning the complete patient's medical image again, and regenerating the surgical guideline marking layer 1 according to an operation requirements; if not, go to step S613, using the currently scannable patient's medical image, and updating by a computer vision calculations to regenerate the surgical guideline marking layer 1. Specifically, if the currently available medical images can fully reconstruct the patient information, the user can decide whether to redesign the surgical information on the surgical guideline marking layer 1 according to the needs and the current situation. If the surgical guideline marking layer 1 is not necessary to redesign just only need to update part of the surgical information on the original surgical guideline marking layer 1, then go to step S613. And if surgical guideline marking layer 1 is necessary to redesign the surgical information on the surgical guideline marking layer 1, then go to step S612, scan the patient image again, and then regenerate the surgical guideline marking layer 1 according to the process in FIG. 25 and the corresponding paragraphs.

It should be understood that the placement of the surgical guideline marking layer 1 above is not limited to a specific order, the placement order can be fixed steps first to determine whether the surgical guideline marking layer 1 is attached or not, and then the body positioning mark 205 is used to determine the placement position; or the body positioning mark 205 is used to determine the placement position and fixing steps first, and then the user can determine whether the surgical guideline marking layer 1 is attached; or the body positioning mark 205 is used to determine the placement position first, the user determine whether the surgical guideline marking layer 1 is attached, and then perform the fixing steps; or the body positioning mark 205 is used to determine the placement position and the fixing step at the same time, and then determine the attached at the end; or, the fixing steps, the determination of the attached degree, and body positioning mark 205 is used to determine the placement position at the same time.

The present disclosure further provides a system for producing a surgical guideline marking layer 1 including a disposing device and marking device. The disposing device is configured to dispose the body positioning mark on at least one layer, as a reference for disposing at least one layer of object surface features on the object surface. The marking device is configured to dispose the surgical guideline mark on at least one layer as a guiding reference for the surgical path, so as to obtain the surgical guideline marking layer 1.

In some embodiments, the system further includes the fixing device configured to perform at least one layer of fixing ways, the fixing ways includes disposing adhesive under at least one layer, disposing a fixing device under at least one layer, disposing foldable wire above or below at least one layer, or disposing at least one receiving tank on the periphery of at least one layer.

In some embodiments, the body positioning mark is attaching device, surgical guideline mark, predetermined position, object surface features, second continuous grid, second discontinuous grid having at least one blank, at least one blank mark, at least one conductive blank correspondingly dispose to the at least one blank, or a combination thereof.

In some embodiments, the disposing device is further configured to dispose the medical image imageable mark on the object surface, and the curved surface of the object surface is reconstructed through computer vision calculation, in which, the medical image imageable mark includes the first continuous grid, the first discontinuous grid having at least one blank, or a combination thereof; and the system further includes a photography device configured to scan the medical image together with the medical image imageable mark and the object on the object surface to generate the surgical guideline marking layer including the object surface features, the second continuous grid, the second discontinuous grid having at least one blank, the at least one blank mark, or a combination of surgical guideline marking layer.

In some embodiments, the disposing device is further configured to dispose the medical image imageable mark on the object surface, and the curved surface of the object surface is reconstructed through computer vision calculation, in which, the medical image imageable mark includes the first discontinuous grid having at least one blank; and the system further includes the photography device configured to scan medical images together with the medical image imageable mark and the object on the object surface to generate the surgical guideline marking layer including the surface features of the object and at least one blank mark, in which, the surgical guideline marking layer further includes at least one conductive blank mark is correspondingly disposed on at least one blank, and the surgical guideline marking layer is disposed under the surgical guideline marking layer.

In some embodiments, the body positioning mark is printing ink or medical image imageable mark, in which, the body positioning mark includes circles, polygons, or physiological characteristics.

In some embodiments, the marking device is more configured to place the medical image imageable mark on the object surface; the system further includes the photography device configured to scan the object surface with the medical image imageable mark, in which, when the medical image imageable mark is printed on at least one layer, the medical image imageable mark becomes the body positioning mark.

In some embodiments, the disposing device is further configured to dispose auxiliary information in at least one layer, in which, the auxiliary information includes patient medical images, patient identification data, disease reminder, surgical reminder information, or a combination thereof on at least one layer.

In some embodiments, the surgical guideline mark includes visual features, tactile features, physical object features, or a combination thereof.

In some embodiments, the visual features further include circles, polygons, lines, pinheads, fillets, the surgical instrument copy shape, an implant copy shape, at least one optical refraction, reflection, and light-focusing effect grating structure, or a combination thereof.

In some embodiments, the tactile feature further includes at least one directional protruded structure.

In some embodiments, the physical object characteristic includes the fixing device.

In some embodiments, the system for producing the surgical guideline marking layer further includes the robotic arm configured to fix the surgical guideline marking layer on the object surface, and the robotic arm is configured to place the medical image imageable mark or the surface feature of the object on the object surface. The surgical guideline marking layer is positioned after the body positioning mark corresponding to the surgical guideline marking layer.

The present disclosure also provides a method for producing the surgical guideline marking layer system, including: a disposing device is used to dispose the body positioning mark on the surgical guideline marking layer first, as a reference for disposing the surgical guideline marking layer on the predetermined position of the object surface; then the marking device is used to dispose the surgical guideline mark on the surgical guideline marking layer as a guideline for the surgical path.

While the present disclosure has been described by way of example(s) and in terms of the preferred embodiment(s), it is to be understood that the disclosure is not limited thereto. On the contrary, it is intended to cover various modifications and similar arrangements and procedures, and the scope of the appended claims therefore should be accorded the broadest interpretation so as to encompass all such modifications and similar arrangements and procedures.

1. A bi-layer surgical guideline marking layer, comprising:
a first layer being transparent material;
a second layer being transparent material, and the second layer relatively disposed under the first layer; and
a first receiving tank disposed on a first same side of the first layer and the second layer.

2. The bi-layer surgical guideline marking layer of claim 1, further comprising a second receiving tank disposed on a second same side of the first layer and the second layer, and disposed at an opposite side of the first receiving tank, an adjacent side of the first receiving tank, or the opposite side and two adjacent sides of the first receiving tank.

3. The bi-layer surgical guideline marking layer of claim 1, further comprising:
a first permeable membrane disposed between the first same side and the first receiving tank; and
an access hole disposed on the first layer.

4. The bi-layer surgical guideline marking layer of claim 2, further comprising:
a first permeable membrane disposed between the first same side and the first receiving tank;
a second permeable membrane disposed between the second receiving tank, and the first layer and the second layer; and
an access hole disposed on the first layer.

5. The bi-layer surgical guideline marking layer of claim 1, wherein the first layer displays a first color, the second layer displays a second color, the first color is different from the second color, when the first layer overlaps the second layer, the first layer displays a third color.

6. The bi-layer surgical guideline marking layer of claim 1, further comprising at least one first instrument through hole penetrating the first layer and the second layer, and the first layer and the second layer closely attached around the at least one first instrument through hole.

7. The bi-layer surgical guideline marking layer of claim 1, further comprising at least one removable structure, at least one hardware adapter, at least one attaching device, at least one tactile structure, at least one grating structure, at least one body positioning mark, or at least one medical image layer disposed on the first layer, under the first layer, on the second layer, under the second layer or a combination thereof.

8. The bi-layer surgical guideline marking layer of claim 1, further comprising a foldable wire disposed at opposite sides or all around of the first layer and the second layer.

9. The bi-layer surgical guideline marking layer of claim 8, further comprising at least one signal element disposed on the foldable wire to sense an attached state of the bi-layer surgical guideline marking layer.

10. A method of producing surgical guideline marking layer, comprising:
providing at least one layer;
a body positioning mark disposed at the at least one layer, as a reference for disposing the at least one layer at an object surface feature on an object surface; and
a surgical guideline mark disposed on the at least one layer, as a guide reference for a surgical path, so as to obtain the surgical guideline marking layer.

11. The method of claim 10, further comprising performing a fixing way of the at least one layer, the fixing way comprising disposing an adhesive under the at least one layer, disposing a fixing device under the at least one layer, disposing a foldable wire on or under the at least one layer, or disposing at least one receiving tank around the at least one layer.

12. The method of claim 10, wherein the body positioning mark is an attaching device, the surgical guideline mark, a predetermining position, the object surface feature, a second continuous grid, a second discontinuous grid having at least one blank, at least one blank mark, at least one conducting blank mark relatively disposed at the at least one blank, or a combination thereof.

13. The method of claim 12, wherein the step of the body positioning mark disposed at the at least one layer, comprising:
disposing a medical image imageable mark on the object surface, and reconstructing a curved surface of the object surface through a computer vision calculation, wherein the medical image imageable mark comprises a first continuous grid, a first discontinuous grid having at least one blank, or a combination thereof; and
scanning a medical imaging for the medical image imageable mark together with an object of the object surface to produce the surgical guideline marking layer comprising the object surface feature, the second continuous grid, the second discontinuous grid having at least one blank, at least one blank mark, or a combination thereof.

14. The method of claim 12, wherein the step of the body positioning mark disposed at the at least one layer, comprises:
disposing a medical image imageable mark on the object surface, and reconstructing a curved surface of the object surface through a computer vision calculation, wherein the medical image imageable mark comprises a first discontinuous grid having at least one blank; and
scanning a medical imaging for the medical image imageable mark together with an object of the object surface to produce the surgical guideline marking layer comprising the object surface feature, and the at least one blank mark,
wherein the first discontinuous grid having at least one blank is a conductive substance;
wherein the surgical guideline marking layer further comprises at least one conducting blank mark relatively disposed at the at least one blank, and disposed under the surgical guideline marking layer.

15. The method of claim 10, wherein the step of the body positioning mark disposed at the at least one layer, comprises printing the body positioning mark on the at least one layer, wherein a material of the body positioning mark is printing ink or a material that to be imaged in medical imaging, wherein the body positioning mark comprises circle, polygon, or physiological characteristics.

16. The method of claim 15, before wherein the step of body positioning mark printed at the at least one layer, comprises:
placing a medical image imageable mark on the object surface; and
scanning the object surface having the medical image imageable mark, wherein the medical image imageable mark printed on at least one layer becomes the body positioning mark.

17. The method of claim 10, further comprising printing auxiliary information on the at least one layer, wherein the auxiliary information comprises a patient's medical image, a patient's identification data, a condition reminder, a surgery reminder information, or a combination thereof.

18. The method of claim 10, wherein the surgical guideline mark comprises a visual characteristic, a tactile characteristic, a physical object characteristic, or a combination thereof;
wherein the visual characteristic comprises a circle, a polygon, a pinhead, a line, a fillet, a surgical instrument copy shape, an implant copy shape, at least one grating structure with optical refraction, reflection, light concentrating effect, or a combination thereof;
wherein the tactile characteristic comprises at least one directional protruded structure;
wherein the physical object characteristic comprises a fixing device.

19. A method of using surgical guideline marking layer, comprising:
providing at least one surgical guideline marking layer obtained by the method of producing as claimed in claim 10;
fixing the surgical guideline marking layer on an object surface; and
corresponding an identification mark or an object surface feature on the object surface to the body positioning mark and/or the surgical guide mark of the surgical guideline marking layer to position the surgical guideline marking layer.

20. The method of claim 19, wherein the step of fixing the surgical guideline marking layer on the object surface, comprises using a fixing device, frictional force or static electricity of the surgical guideline marking layer, adhesive, or a foldable wire way to fix the surgical guideline marking layer on the object surface.

21. The method of claim 19, wherein the step of corresponding the identification mark or the object surface feature on the object surface to the body positioning mark and/or the surgical guide mark of the surgical guideline marking layer comprises:
printing a predetermining position of the body positioning mark and the object surface feature on the surgical guideline marking layer, corresponding the object surface feature on the surgical guideline marking layer to the object surface feature on the object surface, and then disposing the body positioning mark at the predetermining position;
scanning the object surface to obtain a medical image; and
determining whether a distance between the body positioning mark in the medical image and the object surface feature in the medical image is the same as the distance between the body positioning mark placed on the surgical guideline marking layer and the object surface feature on the surgical guideline marking layer, if they are not the same, adjust the position of the surgical guideline marking layer until the same.

22. The method of claim 19, wherein the step of corresponding the identification mark or the object surface feature on the object surface to the body positioning mark and/or the surgical guide mark of the surgical guideline marking layer comprises: printing the object surface feature on the surgical guideline marking layer, and corresponding the object surface feature on the surgical guideline marking layer to the object surface feature on the object surface.

23. The method of claim 19, wherein the step of corresponding the identification mark or the object surface feature on the object surface to the body positioning mark and/or the surgical guide mark of the surgical guideline marking layer comprises: after scanning a medical image imageable mark image on the object surface to formed the body positioning mark on the surgical guideline marking layer; before removing the medical image imageable mark, marking the position of the medical image imageable mark on the object surface by using the identification mark.

24. The method of claim 19, further comprising determining an attached degree of the surgical guideline marking layer on the object surface.

25. The method of claim 24, wherein the step of determining the attached degree of the surgical guideline marking layer on the object surface, comprises using at least one grating structure disposed on the surgical guideline marking layer, wherein when the surgical guideline marking layer is completely attached to the object surface, the body positioning mark, the surgical guideline mark, or a combination thereof will be completely displayed by illuminating the grating structure with light.

26. The method of claim 24, wherein the step of determining the attached degree of the surgical guideline marking layer on the object surface, comprises:
disposing a medical image imageable mark on the object surface, and reconstructing a curved surface of the object surface through a computer vision calculation, wherein the medical image imageable mark comprises a first continuous grid, a first discontinuous grid having at least one blank, or a combination thereof;
scanning a medical imaging for the medical image imageable mark together with an object of the object surface to produce the surgical guideline marking layer comprising a patient's medical image, a second continuous grid, a second discontinuous grid having at least one blank, at least one blank mark, or a combination thereof; and
disposing the surgical guideline marking layer on the object surface,
wherein, when the second continuous grid is corresponding to the first continuous grid, the surgical guideline marking layer is determined to attach to the object surface;
wherein, when the second discontinuous grid having at least one blank is corresponding to the first discontinuous grid having at least one blank, the surgical guideline marking layer is determined to attach to the object surface; or
wherein, when the at least one blank mark is corresponding to the first discontinuous grid having at least one blank, the surgical guideline marking layer is determined attach to the object surface.

27. The method of claim 24, wherein the step of determining the attached degree of the surgical guideline marking layer on the object surface, comprises:
disposing a medical image imageable mark on the object surface, and reconstructing a curved surface of the object surface through a computer vision calculation, wherein the medical image imageable mark comprises a first discontinuous grid having at least one blank;
scanning a medical imaging for the medical image imageable mark together with an object of the object surface to produce the surgical guideline marking layer comprising a patient's medical image, a second discontinuous grid having at least one blank, at least one blank mark, or a combination thereof; and
disposing the surgical guideline marking layer on the object surface,
wherein the first discontinuous grid having at least one blank is a conductive substance;
wherein the surgical guideline marking layer further comprises at least one conductive blank mark correspondingly disposed at the at least one blank, and disposed under the surgical guideline marking layer;
wherein, when the at least one conductive blank mark is electrically connected to the first discontinuous grid having the at least one blank, the surgical guideline marking layer is determined to attach to the object surface.

28. The method of claim 27, wherein the at least one conductive blank mark comprises a first distance sensor communicating with a second distance sensor on a surgical instrument.

29. The method of claim 19, further comprising determining whether a surgical information on the surgical guideline marking layer is consistent with a current state of a patient, if so, starting to use the surgical guideline marking layer.

30. The method of claim 29, wherein the step of determining whether a surgical information on the surgical guideline marking layer is consistent with the current state of the patient, if not,
determining whether the object surface remains a medical image imageable mark produced in a previous surgical guideline marking layer, if not, regenerating another surgical guideline marking layer according to surgical requirements.

31. The method of claim 30, wherein the step of determining whether the object surface remains a medical image imageable mark produced in the previous surgical guideline marking layer, if so,
determining whether a complete patient's medical image can be scanned at the moment, if not, regenerating the surgical guideline marking layer by scanning a current medical image of the patient, and using a computer vision calculations to update the medical image of the patient.

32. The method of claim 31, wherein the step of determining whether a complete patient's medical image can be scanned at the moment, if so,
determining whether the surgical information on a surgical guideline marker layer is necessary to redesign, if so, scanning the complete patient's medical image again, and regenerating the surgical guideline marking layer according to an operation requirements; if not, then using a currently scannable patient's medical image, and updating by a computer vision calculations to regenerate the surgical guideline marking layer.
